(19) United States
(12) Patent Application Publication
(10) Pub. No.: US 2025/0230381 A1
(43) Pub. Date: Jul. 17, 2025

(54) METHOD OF PRODUCING VEGETABLE MIXED SURFACTANTS UNDER ALKALINE CONDITIONS, IMPROVING LONG-TERM STABILIZATION, AND COSMETIC COMPOSITION USING THE SAME

(71) Applicant: BIOBEAUTECH Co., Ltd., Seongam-si (KR)

(72) Inventors: In Young Kim, Seongnam-si (KR); Jae Young Yeon, Yongin-si (KR)

(73) Assignee: BIOBEAUTECH CO., LTD., Seongam-si (KR)

(21) Appl. No.: 18/754,770

(22) Filed: Jun. 26, 2024

(30) Foreign Application Priority Data
Jan. 11, 2024 (KR) 10-2024-0004989

(51) Int. Cl. C11C 3/00; A61K 8/02; A61K 8/37; A61Q 19/08

(52) U.S. Cl. CPC C11C 3/003; A61K 8/0291; A61K 8/375; A61Q 19/08

(57) ABSTRACT

Disclosed is a vegetable mixed surfactant that has both solubilizing and emulsifying power based on a combination of high-purity polyglyceryl-10 oleate and polyglyceryl-10 stearate without adding ethylene oxide. In this case, the vegetable mixed surfactant is capable of excellent esterification reaction because it contains arginine and secures long-term stability without causing changes in odor or color because it contains tranexamic acid. The vegetable mixed surfactant has excellent skin stability, very low cytotoxicity, excellent short-term moisturizing effect and long-term moisturizing effect, and superior skin improvement effects such as fine wrinkle amelioration effect, skin whitening effect, and skin lifting improvement effect. The vegetable mixed surfactant may be applied to cosmetic compositions of various formulations (skin toner, hair toner, transparent essences, emulsions, creams, and the like).

### CROSS-REFERENCE TO RELATED APPLICATIONS

This application claims priority to Korean Patent Application No. 10-2024-0004989 filed Jan. 11, 2024, the entire disclosures of which are incorporated herein by reference.

### BACKGROUND OF THE INVENTION

### Field of the Invention

The present invention relates to a method of preparing a vegetable mixed surfactant for improving long-term stability and a cosmetic composition containing the same. More specifically, the present invention relates to a vegetable mixed surfactant with excellent performance synthetized using polyglyceryl as a hydrophilic moiety, and using a mixture of oleic acid and stearic acid as a lipophilic moiety, without adding ethylene oxide, and using arginine to create alkaline conditions in the synthesis process, wherein the vegetable mixed surfactant is imparted with secure long-term stability by adding tranexamic acid, and a cosmetic composition prepared using the resulting surfactant.

### DESCRIPTION OF THE RELATED ART

Common raw materials used as solubilizers in the cosmetic industry include PEG-60 hydrogenated castor oil, PEG-40 hydrogenated castor oil, Polysorbate-20, Glycereth-25 PCA isostearate, and the like and are used to dissolve fragrances, oils or oil-soluble active ingredients in water. Common raw materials used as emulsifiers include Glycereth-25 PCA isostearate, Ceteareth-10 to Ceteareth-30, which are formed by an ether bond of ethylene oxide and fatty acid, surfactant mixed with PEG-100 stearate, and surfactants such as polysorbate-60. These ingredients are mainly used to prepare emulsified formulations such as emulsified lotions, creams, and essences.

Recently, surfactants mixed with cetearyl olivate and sorbitan olivate, and vegetable emulsifiers such as decyl glucoside and coco betaine have been developed and reported. However, these substances function to aid to emulsifiers without a solubilization function, and have lower stability than conventional surfactants. Conventional surfactants not supplemented with ethylene oxide, which are called “vegetable surfactants”, are used as emulsion stabilizers or simply utilized limitedly in topical application in cleansing creams, hair shampoos, and the like. Especially, vegetable surfactants that exhibit both solubilization ability and emulsification ability are rare, and vegetable surfactants have problems with long-term stability due to browning and severe off-flavor.

In order to solve these problems, specific conditions are required for preparing novel high-performance vegetable surfactants with both solubilizing and emulsifying abilities, and stabilizers are increasingly required to ensure long-term stability. There is a need for development of multifunctional, high-purity vegetable surfactants applicable to emulsions, creams, sunblock creams, and hair creams that can be easily emulsified when raw materials for solubilizing beneficial ingredients so as to be clear are used in combination with a high content of oil to prepare formulations such as skin toner.

### PRIOR ART LITERATURE

### Patent Document

(Patent Document 1) Korean Patent No. 10-1082941 (registered on Nov. 7, 2011) relates to a composition with fragrance and antibacterial activity for alleviating or preventing static electricity containing a herbal essential oil and a vegetable surfactant as active ingredients, wherein the composition with fragrance and antibacterial activity improves the anti-static effect based on the combination of the herbal essential oil and polysorbate.
(Patent Document 2) Korean Patent No. 10-2072297 (registered on Jan. 23, 2020) relates to a cosmetic composition that contains a vegetable protein-derived surfactant, a hydrolyzed protein, and a human stem cell culture medium as active ingredients and is thus remarkably effective in improving skin elasticity, skin moisturizing, and ameliorating skin wrinkles based on synergistic effects thereof.

### SUMMARY OF THE INVENTION

In order to solve the problems of the prior art as described above, it is one object of the present invention to provide a vegetable mixed surfactant with excellent performance synthetized using polyglyceryl as a hydrophilic moiety, and using a mixture of oleic acid and stearic acid as a lipophilic moiety, without adding ethylene oxide, and using arginine to create alkaline conditions in the synthesis process, to secure long-term stability by adding tranexamic acid to the surfactant, and to prepare a cosmetic composition using the resulting surfactant.

In accordance with one aspect of the present invention, provided is a method of preparing a vegetable mixed surfactant including dissolving a mixture of oleic acid and stearic acid as a lipophilic moiety in polyglyceryl-10 as a hydrophilic moiety (a), adding arginine to the resulting product in the presence of nitrogen to induce esterification at a pH of 9 to 12.5 (b), adjusting the pH to 6 to 8 using a pH adjuster (c), and stirring the result along with a stabilizer (d).

Meanwhile, in step (b), the esterification is preferably performed twice.

Meanwhile, the stabilizer preferably includes at least one selected from tranexamic acid, tocopheryl acetate, ferulic acid, catechin, and baicalin.

In accordance with another aspect of the present invention, provided is a micelle formed by self-assembly of the vegetable mixed surfactant obtained by the method.

Meanwhile, the micelle preferably has a solubilizing power to provide a transparent phase and an emulsifying power.

Meanwhile, the micelle preferably contains a lipophilic substance trapped therein.

In accordance with another aspect of the present invention, provided is a cosmetic composition containing the micelle.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram illustrating a process of manufacturing a vegetable mixed surfactant of the present invention;

FIG. 2 shows a molecular structure of the synthetized vegetable mixed surfactant of the present invention;

FIG. 3 is an image showing the synthetized vegetable mixed surfactant (Example 3) of the present invention;

FIG. 4 is a diagram illustrating the mechanism of solubilization of the vegetable mixed surfactant of the present invention;

FIG. 5 is a table showing HLB (hydrophilic lipophilic balance) suitable for each application;

FIG. 6 is a graph showing the surface tension of the vegetable mixed surfactant of the present invention;

FIG. 7 is an image illustrating the solubilization mechanism of the present invention;

FIG. 8 is an image showing a transparent solubilized product using the vegetable mixed surfactant of the present invention;

FIG. 9 is an image showing the results of solubilization experiment of vegetable mixed surfactants according to Control group and the present invention;

FIG. 10 is a graph showing the transmittance measured with a UV spectrophotometer to compare solubilizing power between the vegetable mixed surfactants of the present invention and Comparative Examples;

FIG. 11 is an image showing an example in which lavender oil is solubilized using the vegetable mixed surfactant of the present invention;

FIG. 12 is a schematic diagram illustrating a process of manufacturing an emulsion cream containing the vegetable mixed surfactant of the present invention;

FIG. 13 is an image illustrating an emulsion cream containing the vegetable mixed surfactant of the present invention;

FIG. 14 is a micrograph showing the particle diameter of an emulsion cream containing the vegetable mixed surfactant of the present invention;

FIG. 15 is a micrograph showing the particle diameter of an anti-aging cream containing the vegetable mixed surfactant of the present invention;

FIG. 16 is a graph showing the skin stability of the vegetable mixed surfactant of the present invention;

FIG. 17 is a graph showing the results of evaluating the cytotoxicity of the vegetable mixed surfactant of the present invention;

FIG. 18 is a graph showing the results of evaluating the short-term skin moisturizing effect of the vegetable mixed surfactant of the present invention;

FIG. 19 is a graph showing the results of evaluating the long-term skin moisturizing effect of the vegetable mixed surfactant of the present invention;

FIG. 20 is a graph showing the result of evaluating the skin whitening effect of the vegetable mixed surfactant of the present invention;

FIG. 21 is a graph showing the result of evaluating the effect of the vegetable mixed surfactant of the present invention on ameliorating fine skin wrinkles; and

FIG. 22 is a graph showing the result of evaluating skin lifting effect of the vegetable mixed surfactant of the present invention.

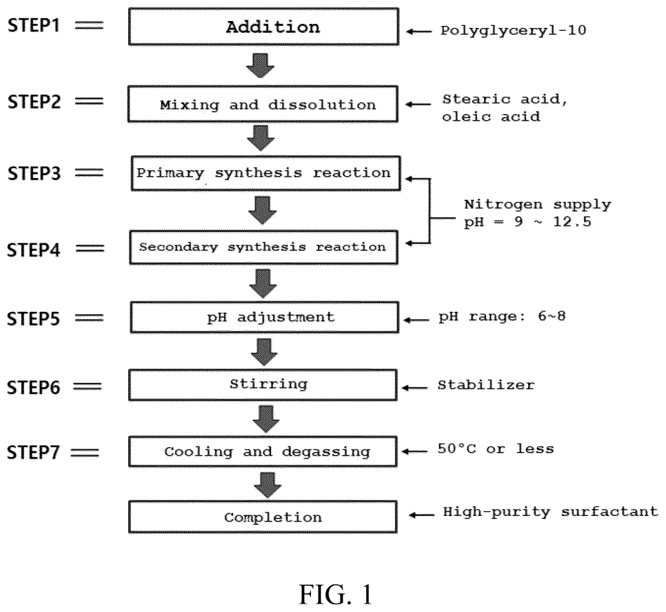

### DETAILED DESCRIPTION OF THE INVENTION

Although attempts were made to develop vegetable surfactants to replace conventional surfactants, conventional vegetable surfactants have problems of low long-term stability due to the limitation to topical application, browning in color, and severe off-flavor. Accordingly, there has been a need for a novel vegetable-derived surfactant having high performance and long-term stability. Therefore, the present invention aims at developing a novel vegetable surfactant that has excellent solubilizing and emulsifying power and various skin functionalities.

The present invention is directed to a method of preparing a vegetable mixed surfactant including dissolving oleic acid and stearic acid as lipophilic moieties in polyglyceryl-10 as a hydrophilic moiety (a), adding arginine to the resulting solution in the presence of nitrogen to induce esterification at a pH of 9 to 12.5 (b), adding a pH adjuster to the result to adjust the pH to 6 to 8 (c), and adding a stabilizer to the result, followed by stirring (d).

In order to develop a high-quality vegetable surfactant, an esterification reaction between high-purity polyglyceryl-10, and oleic acid and stearic acid was performed. Rather than simply synthesizing the hydrophilic and lipophilic moieties, a surfactant with high purity and stability was designed. Accordingly, the best high-purity surfactant could be obtained when synthesized under alkaline conditions (preferably pH=9 to 12.5) using arginine as a catalyst. Such a surfactant was prepared and present as a mixture. In addition, a stabilizer was used to ensure long-term stability, an appropriate stabilizer was identified from among several candidate raw materials, and the product remained stable without off-odor or discoloration while maintaining significantly better stability even during long-term storage.

Meanwhile, in the preparation method of the present invention, polyglyceryl-10 in step (a) may be derived from various vegetable raw materials, but is preferably derived from coconut trees, palm trees, or olive trees. The oleic acid and stearic acid in step (a) may be various vegetable raw materials, and are preferably derived from coconut trees, palm trees, olive trees and the like.

Meanwhile, in the preparation method of the present invention, in step (a), a combination of 40 to 60% by weight of polyglyceryl-10 oleate, in which oleic acid is bound to polyglyceryl-10, and 40 to 60% by weight of polyglyceryl-10 stearate in which stearic acid is bound to polyglyceryl-10 are preferably used, and a combination of 44% by weight of polyglyceryl-10 oleate and 56% by weight of polyglyceryl-10 stearate is more preferably used. By performing synthesis under these conditions, a surfactant with excellent solubilizing and emulsifying power can be obtained. The head of the hydrophilic moiety of polyglyceryl-10 (molecular weight of 895 g/mol) was highly purified, and the tail thereof was esterified with oleic acid (C₁₈H₃₄O₂, molecular weight of 282.469 g/mol) and stearic acid (C₁₈H₃₆O₂, molecular weight of 284.48 g/mol) to impart surfactant function thereto. The prepared raw material is used to prepare a multifunctional vegetable surfactant with excellent solubilizing and emulsifying properties and excellent moisturizing power. In terms of calculation of the molar ratio, the mixing ratio of polyglyceryl-10 and stearic acid (or oleic acid) is set at a molar ratio of 1:1 to obtain polyglyceryl-10 stearate and polyglyceryl-10 oleate, respectively. Specifically, polyglyceryl-10 oleate was prepared from 895 g of polyglyceryl-10 equivalent to 1 mole, and 282.469 g of oleic acid equivalent to 1 mole and polyglyceryl-10 stearate was prepared from 895 g of polyglyceryl-10 equivalent to 1 mole and 284.48 g of stearic acid equivalent to 1 mole.

Meanwhile, after mixing the hydrophilic and lipophilic moieties, the synthesis reaction is preferably performed using arginine as a catalyst in step (b) under a nitrogen atmosphere at pH 9 to 12.5, more preferably at pH 10.5 to 11.5 (most preferably pH 10.8). As a result, a high purity surfactant can be obtained by sufficiently performing reaction so that no fatty acid residue remains.

At this time, the arginine is preferably added at 1.2% by weight based on the total weight. In addition, at this time, the synthesis reaction is performed by inducing an esterification reaction twice. The additional secondary reaction is necessary to remove substances that may remain unreacted during the primary reaction. Preferably, the primary reaction is performed for 2 to 8 hours, the secondary reaction is performed for 1 to 5 hours, and the reaction temperature is preferably 180 to 250° C. More preferably, the primary reaction is performed for 3 to 5 hours, the secondary reaction is performed for 2 to 4 hours, and the reaction temperature is preferably 185 to 230° C. Although the reaction time may vary slightly depending on the production amount within the ranges, synthesis should be performed under these conditions. As a result, an optimal synthesis reaction may be obtained.

Meanwhile, in the preparation method of the present invention, the pH is adjusted using a pH adjuster added during step (c), and the pH adjuster is any one known in the art without limitation and is preferably lactic acid or citric acid. Preferably, the synthesis is completed at a pH of 6 to 8. When the pH is out of the range defined above, the solubilizing and emulsifying power may decrease, thus deteriorating the viscosity, and causing emulsion phase separation and inconvenient pH adjustment process when preparing emulsion cosmetics using the surfactant. For this reason, the pH is preferably within the range.

Meanwhile, in the preparation method of the present invention, a stabilizer was added in step (d) to improve the long-term stability of the vegetable mixed surfactant of the present invention. The stabilizer may be any one known in the art without limitation. The stabilizer preferably includes at least one selected from tranexamic acid, tocopheryl acetate, ferulic acid, catechin, and baicalin, more preferably, tranexamic acid. In this case, a vegetable mixed surfactant with excellent solubilizing and emulsifying power and excellent long-term stability can be obtained under the conditions defined above.

Meanwhile, in the preparation method of the present invention, the vegetable mixed surfactant may be completed through cooling and degassing after step (d). In this case, the cooling and degassing are preferably performed at 50° C. or lower. The prepared vegetable mixed surfactant has sufficient solubilizing power because the HLB (hydrophilic/lipophilic balance) falls within the range of 14.8 to 15.5, and this composition has the surface tension of 1.100×10⁻⁴ to 1.500×10⁻² dyne/cm (average of 1.2551×10⁻³ dyne/cm) and thus has an optimal critical concentration at low concentrations and sufficient solubilizing power even at low concentrations.

In addition, the present invention provides micelles formed by self-assembly of the vegetable mixed surfactant obtained by the preparation method. Meanwhile, the micelles may preferably have transparent solubilizing power and emulsifying power. Also, at this time, the micelle preferably contains a lipophilic substance trapped therein.

In addition, the present invention provides a cosmetic composition containing the micelles.

Meanwhile, in the cosmetic composition of the present invention, the cosmetic composition may be used to improve skin wrinkles, whiten skin, improve skin elasticity, or moisturize skin.

Meanwhile, in the present invention, the cosmetic composition includes, for example, at least one of skin care formulations selected from solutions, suspensions, emulsions, pastes, cosmetic water, gels, water-soluble liquids, creams, essence, surfactant-containing cleansing, oils, and oil-in-water (O/W)- and water-in-oil (W/O)-type formulations; skin toner; lotions; eye creams; soothing gels; ointments; formulations for mask packs; formulations for body wash; peeling gels; oil-in-water and water-in-oil makeup bases; foundations; skin covers; color makeup formulations selected from lipsticks, lip glosses, face powders, two-way cakes, eye shadows, cheek colors, and eyebrow pencils; and formulations for scalps. Preferably, the cosmetic composition includes one or more formulations selected from the group consisting of transparent skin toner, transparent essence, low-viscosity emulsion lotion essence, and high-viscosity emulsion cream. More preferably, the cosmetic composition is skin toner, ampoule, low-viscosity emulsion, essence, cream, eye cream, sunscreen, makeup base, cushion BB, sheet mask pack, cleanser, lipstick, shampoo for protecting hair, rinse or the like.

In addition, the cosmetic composition of the present invention is used in combination with another cosmetic composition. In addition, the cosmetic composition according to the present invention may be used in an ordinary manner, and the number of times of use may be varied depending on the skin condition or preference of the user.

Meanwhile, according to the following experiment, the present invention developed a vegetable mixed surfactant that contains a mixture of polyglyceryl-10 oleate and polyglyceryl-10 stearate and thus exhibits both solubilizing and emulsifying power. In this case, arginine was used to provide excellent esterification and tranexamic acid was used to secure long-term stability without changes in odor or color. The vegetable mixed surfactant of the present invention is capable of solubilizing fragrances and of emulsifying various oils, and exhibits excellent skin stability, very low cytotoxicity, excellent short-term and long-term moisturizing effects, and superior skin improvement effects such as fine wrinkle relieving, whitening, and lifting effects, and thus can be applied to cosmetic compositions of various formulations (such as skin toner, hair toner, transparent essence, emulsion, and cream).

Hereinafter, the present invention will be described in more detail with reference to the following examples and experimental examples, but the scope of the present invention is not limited to the examples and experimental examples, and includes variations and technical concepts equivalent thereto.

### Examples 1 to 24: Preparation of Vegetable Mixed Surfactant Under Alkaline Conditions and Measurement of HLB, Surface Tension, and Solubilization Power Thereof

In this example, a vegetable mixed surfactant was prepared under alkaline conditions, and the HLB, surface tension, and solubilization power thereof were measured.

### 1) Production of Vegetable Surfactant Under Alkaline Conditions

As shown in Table 1 below, polyglyceryl-10 stearate and polyglyceryl-10 oleate were mixed under different conditions in various ratios based on 100% of the total weight, the pH is adjusted to an alkalinity using arginine, tranexamic acid, tocopheryl acetate, ferulic acid, catechin, and baicalin were used as candidate substances for stabilizers to prepare vegetable mixed surfactants, and solubilization/emulsification power and long-term stability were tested.

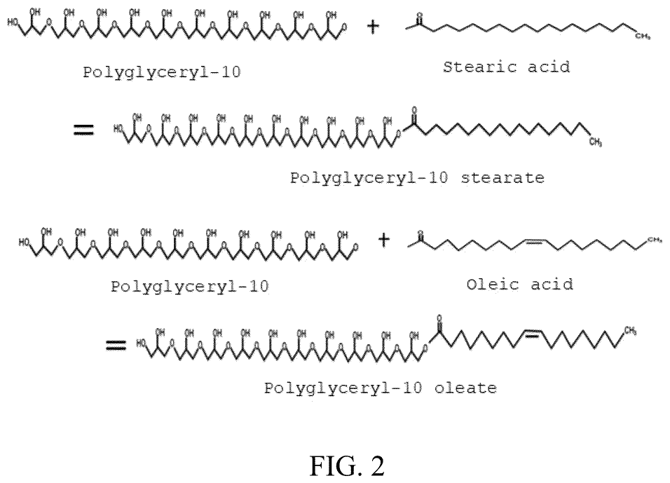

Specifically, a vegetable surfactant was prepared by \textcircled{1} feeding polyglyceryl-10, \textcircled{2} dissolving stearic acid and oleic acid therein while mixing, \textcircled{3} performing primary synthesis reaction using arginine (1.2% by weight) under nitrogen atmosphere at pH of 10.8 at 210° C. for 3 to 5 hours, \textcircled{4} performing secondary synthesis reaction under nitrogen atmosphere at 195° C. and at pH of 10.8 for 2 to 4 hours, \textcircled{5} adjusting the pH to 6 to 8 using a pH adjuster (lactic acid or citric acid), \textcircled{6} adding a stabilizer (any one of tranexamic acid, tocopheryl acetate, ferulic acid, catechin, and baicalin) thereto, followed by stirring, \textcircled{7} cooling the reaction product to 50° C. or lower, followed by degassing to prepare a vegetable surfactant. This process diagram is shown in FIG. 2. At this time, the secondary synthesis reaction can be effectively performed only by stirring the ingredients for an additional time at only a controlled temperature due to the presence of arginine added during the primary synthesis reaction.

In the present invention, in order to develop a composition that is an organic or vegetable surfactant and has solubilizing power, first, a hydrophilic moiety and a lipophilic moiety should be selected. The present invention is characterized in that polyglyceryl-10 was selected as the hydrophilic moiety, and stearic acid and oleic acid were selected as the lipophilic moieties. The head of the hydrophilic moiety of polyglyceryl-10 (molecular weight of 895 g/mol) was highly purified, and the tail thereof was esterified with oleic acid (C₁₈H₃₄O₂, molecular weight of 282.469 g/mol) and stearic acid (C₁₈H₃₆O₂, molecular weight of 284.48 g/mol) to impart surfactant function thereto. The prepared raw material was used to develop a multifunctional vegetable surfactant with excellent solubilizing and emulsifying properties and excellent moisturizing power. The mixing ratio of polyglyceryl-10 to stearic acid (or oleic acid) was set at a molar ratio of 1:1 to obtain polyglyceryl-stearate (or polyglyceryl-10 oleate). Specifically, polyglyceryl-10 oleate was prepared from 895 g of polyglyceryl-10 equivalent to 1 mole and 282.469 g of oleic acid equivalent to 1 mole, and polyglyceryl-10 stearate was prepared from 895 g of polyglyceryl-10 equivalent to 1 mole and 284.48 g of stearic acid equivalent to 1 mole.

In order to impart both solubilizing and emulsifying power to the prepared product, 30 to 70% by weight of polyglyceryl-10 stearate and 70 to 30% by weight of polyglyceryl-10 oleate were mixed in various contents and whether or not the product satisfied these performances was determined. As a result, good results were obtained from a combination of 50 to 60% by weight of polyglyceryl-10 stearate and 40 to 50% by weight of polyglyceryl-10 oleate. More specifically, it was found that the combination of 56% by weight of polyglyceryl-10 stearate and 44% by weight of polyglyceryl-10 oleate had the best performance.

As shown in the molecular structure of FIG. 2, polyglyceryl-10 as the hydrophilic moiety forms an ester bond with stearic acid and oleic acid as the tail to produce polyglyceryl-10 stearate and polyglyceryl-10 oleate, respectively. Each was synthesized with caution such that reaction was thoroughly performed to prevent fatty acids from remaining unreacted in order to prepare excellent surfactants. The solubilization mechanism of the surfactant thus obtained is shown in FIG. 4 for better illustration.

TABLE 1

| | Exam- | Exam- | Exam- | Exam- | Exam- | Exam- |
|---|---|---|---|---|---|---|
| Component | ple 1 | ple 2 | ple 3 | ple 4 | ple 5 | ple 6 |
| name | (wt %) | (wt %) | (wt %) | (wt %) | (wt %) | (wt %) |
| Polyglyceryl-10 | 50 | 60 | 56 | 40 | 70 | 30 |
| stearate | | | | | | |
| Polyglyceryl-10 | 50 | 40 | 44 | 60 | 30 | 70 |
| oleate | | | | | | |
| Total | 100 | 100 | 100 | 100 | 100 | 100 |
| Arginine | 1.2 | 1.2 | 1.2 | 1.2 | 1.2 | 1.2 |
| Tranexamic acid | — | — | 0.1 | — | — | — |
| Tocopheryl | — | 0.1 | — | — | — | — |
| acetate | | | | | | |
| Ferulic acid | 0.1 | — | — | — | — | — |
| Catechin | — | — | — | 0.1 | — | — |
| Baicalin | — | — | — | — | — | 0.1 |
| Solubilizing | Poor | Good | Good | Good | Poor | Poor |
| power/ | | | | | | |
| emulsifying | | | | | | |
| power | | | | | | |
| Long-term | Change | Change | Good | Change | Change | Change |
| stability | in | in | | in | in | in |
| | odor | color | | color | color | color |

*Solubilization-phase test method: whether the appearance of the formed micelles was suspended or transparent when 1 g of each Example is dispersed in 100 g of water was determined.
*Emulsion-phase test method: 1 g of mixed surfactant was mixed with 20 g of mineral oil, the mixture was heated to 80° C. and then mixed with purified water, followed by stirring for 3 minutes at 3,500 rpm with a homogenizer. The resulting appearance was observed by the naked eye.

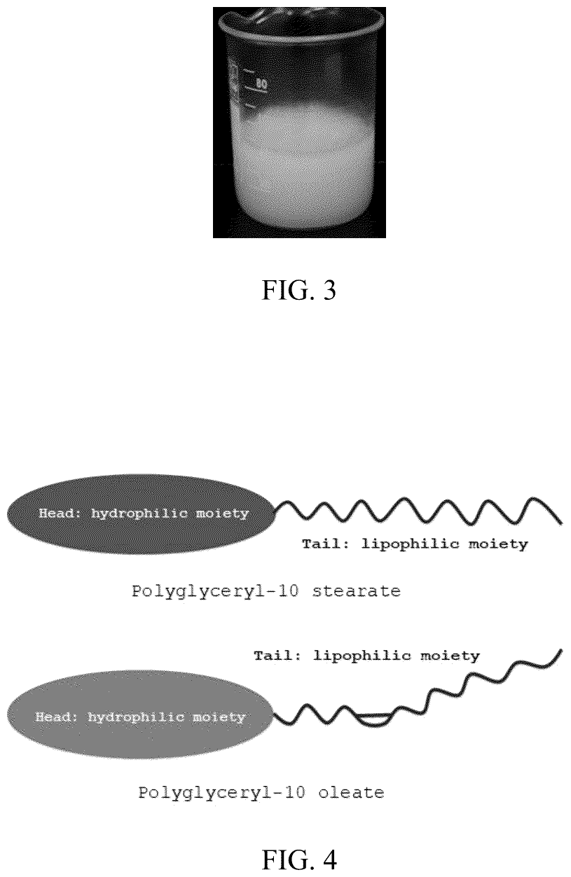

As a result, the vegetable surfactant prepared by mixing polyglyceryl-10 stearate and polyglyceryl-10 oleate in the ratio of Example 3, mixing with 1.2% by weight of arginine, and mixing with 0.1% by weight of tranexamic acid as a stabilizer exhibited excellent solubilization/emulsification power and high long-term stability. The sample thus obtained was a light yellow paste (FIG. 3), had a slight inherent odor a specific gravity of 1.15, and a very low acid value of 0.072±0.1, which indicates that a complete reaction occurred.

Then, arginine (1.2% by weight) was used as a reaction catalyst above, tranexamic acid (0.1% by weight), which was an appropriate stabilizer was used, and polyglyceryl-10 stearate and polyglyceryl-10 oleate were mixed in various ratios based on 100% of the total weight (Table 2) to prepare vegetable surfactants under different conditions, and then solubilization phase, emulsion phase and long-term stability thereof were tested.

TABLE 2

| | Exam- | Exam- | Exam- | Exam- | Exam- | Exam- |
|---|---|---|---|---|---|---|
| | ple | ple | ple | ple | ple | ple |
| Component | 7 | 8 | 9 | 10 | 11 | 12 |
| name | (wt %) | (wt %) | (wt %) | (wt %) | (wt %) | (wt %) |
| Polyglyceryl- | 50 | 60 | 56 | 40 | 70 | 30 |
| 10 stearate | | | | | | |
| Polyglyceryl- | 50 | 40 | 44 | 60 | 30 | 70 |
| 10 oleate | | | | | | |
| Total | 100 | 100 | 100 | 100 | 100 | 100 |
| Arginine | 1.2 | 1.2 | 1.2 | 1.2 | 1.2 | 1.2 |
| Tranexamic | 0.1 | 0.1 | 0.1 | 0.1 | 0.1 | 0.1 |
| acid | | | | | | |
| Solubilization | Suspen- | Suspen- | Suspen- | Suspen- | Suspen- | Suspen- |
| phase | sion | sion | sion | sion | sion | sion |
| | phase | phase | phase | phase | phase | phase |
| Emulsion | Milky | Milky | Milky | Milky | Milky | Milky |
| phase | white | white | white | white | white | white |
| Long-term | X | Δ | ⊚ | ◯ | Δ | X |
| stability | | | | | | |

*Solubilization-phase test method: whether the appearance of the formed micelles was suspended or transparent when 1 g of each Example is dispersed in 100 g of water was determined.
*Emulsion-phase test method: 1 g of mixed surfactant was mixed with 20 g of mineral oil, the mixture was heated to 80° C. and then mixed with purified water, followed by stirring for 3 minutes at 3,500 rpm with a homogenizer. The resulting appearance was observed with the naked eye.
*Long-term stability: after storage in 50° C. incubator for one month, the change in color was observed with the naked eye. (X: Poor, Δ: odor change, but, no color change with separation, ◯: slight change, but no separation, ⊚: all good)

As a result, the surfactant prepared by mixing polyglyceryl-10 stearate and polyglyceryl-10 oleate in the ratio of Example 9 (same as in Example 3, hereinafter referred to as “Example 3”) exhibited excellent solubilization power to provide the most transparent phase. Other Examples exhibit insufficient solubilizing power to provide a suspension phase and a combination of 56% by weight of polyglyceryl-10 stearate with 44% by weight of polyglyceryl-10 oleate exhibits excellent solubilizing power to provide a transparent phase at a specific ratio. In addition, Example 9 exhibited the best long-term stability.

### 2) Measurement of HLB of Vegetable Surfactant of the Present Invention

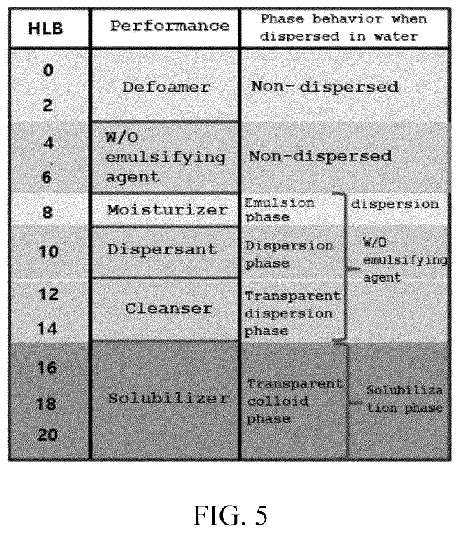

HLB indicates the balance between hydrophilic and lipophilic moieties (hydrophilic-lipophilic balance). As shown in FIG. 5, the vegetable surfactant may be used depending on the HLB according to the function and application. The HLB is calculated by dividing the molecular weight of the hydrophilic moiety by a total of the molecular weight of the hydrophilic moiety and the molecular weight of the lipophilic moiety, and multiplying the result by 100 to obtain a percentage HLB. The HLB percentage value may be divided by 5 to obtain the desired HLB.

The HLB is measured using this calculation method to the present invention as follows: the molecular weight of polyglyceryl-10, which has a hydrophilic moiety, is about 895 g/mol, the molecular weight of oleic acid is 282.468 g/mol, and the molecular weight of stearic acid is 284.48 g/mol. When the two ingredients were 44% oleic acid and 56% stearic acid, the average molecular weight is calculated to be about 283.62 g/mol. The total molecular weight of the final surfactant synthesized from these ingredients is 1,187.62 g/mol. In the end, the molecular weight of the hydrophilic moiety, 895, is divided by 1187.62 to calculate the percentage HLB, 75.36. The percentage HLB is divided by 5, the HLB becomes about 15.072. Therefore, the HLB of the surfactant of the present invention is about 15.1.

In addition, the results of measurements calculated through actual experiments show that the surfactant has an HLB of 15.25, which was similar to the HLB of the surfactant of the present invention, and exhibits excellent solubilizing power. The solubilizing power of a currently commercially available surfactant having an HLB of 15 is measured at the same concentration under the same conditions and is then quantified, which is a conventional method.

Generally, it has been reported that a surfactant having an HLB ranging from 14 to 18 exhibits sufficient solubilizing power and thus the mixed surfactant obtained in the present invention may be considered to be a raw material having sufficient solubilizing power. In addition, the surfactant obtained in the present invention is expected to have excellent skin moisturizing power because it has more —OH groups than conventional surfactants, to have a softening effect due to the combination of organic or vegetable fatty acids, and to be safe for the skin.

### 3) Measurement of Surface Tension of Vegetable Surfactant of the Present Invention

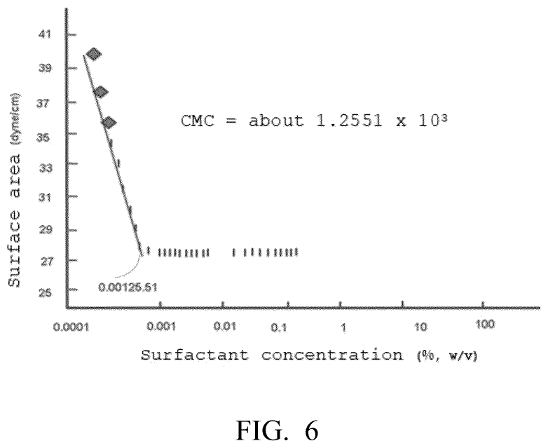

The measurement of the surface tension of the vegetable surfactant (Example 3) of the present invention is performed by dropping a very small amount of the surfactant into water and measuring the optimal critical concentration of micelles formed when the concentration is gradually increased. The surface tension may be an indicator for the performance of solubilizing power. The result of surface tension measurement, as shown in FIG. 6, shows that the surface tension was found to be 1.100×10⁻⁴ to 1.500×10⁻² dyne/cm (mean, 1.2551×10⁻³ dyne/cm). From the graph, it can be seen that the surfactant has an optimal critical concentration at a very low concentration, which means that the vegetable surfactant of the present invention has sufficient solubilizing power even at a very low concentration.

### 4) Measurement of Solubilization Power of Vegetable Surfactant of the Present Invention

To measure the solubilizing power of the vegetable surfactant of the present invention (Example 3), bergamot oil and tocopheryl acetate as oily phases were solubilized (each used at 0.1% by weight) and the solubilizing power of the vegetable surfactant of the present invention was compared with the solubilizing power of PEG-60 hydrogenated castor oil, which is a typical conventional surfactant obtained from the petroleum (Table 3).

TABLE 3

| | | | | | Compar- | Compar- |
|---|---|---|---|---|---|---|
| | | | | | ative | rative |
| | Exam- | Exam- | Exam- | Exam- | Exam- | Exam- |
| | ple | ple | ple | ple | ple | ple |
| Component | 13 | 14 | 15 | 16 | 1 | 2 |
| name | (wt %) | (wt %) | (wt %) | (wt %) | (wt %) | (wt %) |
| Example 3 | 1 | 1.5 | 1 | 1.5 | — | — |
| (vegetable | | | | | | |
| surfactant) | | | | | | |
| PEG-60 | — | — | — | — | 1 | 1 |
| hydrogenated | | | | | | |
| castor oil | | | | | | |
| Bergamot oil | 0.1 | 0.1 | — | — | 0.1 | — |
| (oily phase) | | | | | | |
| Tocopheryl | — | — | 0.1 | 0.1 | — | 0.1 |
| acetate | | | | | | |
| (oily phase) | | | | | | |
| Purified water | 98.9 | 98.4 | 98.9 | 98.4 | 98.9 | 98.9 |
| Total | 100 | 100 | 100 | 100 | 100 | 100 |
| Appearance | Trans- | Trans- | Trans- | Trans- | Trans- | Trans- |
| | parent | parent | parent | parent | parent | parent |
| | liquid | liquid | liquid | liquid | liquid | liquid |
| pH (10% | 6.32 | 6.46 | 6.28 | 6.12 | 6.46 | 6.51 |
| aqueous | | | | | | |
| solution) | | | | | | |
| gravity | 1.005 | 1.012 | 1.008 | 1.002 | 1.003 | 1.026 |
| Stability | Stable | Stable | Stable | Stable | Stable | Stable |
| (after 4 | | | | | | |
| weeks at | | | | | | |
| 45° C.) | | | | | | |

*Preparation method: 1) The vegetable surfactant of Example 3 or the surfactant of Comparative Example, bergamot oil and tocopheryl acetate were weighed, followed by heating to 80° C..
2) Purified water (aqueous phase) was slowly added to the result while rotating at 20-100 rpm, and the solubilization state was compared.

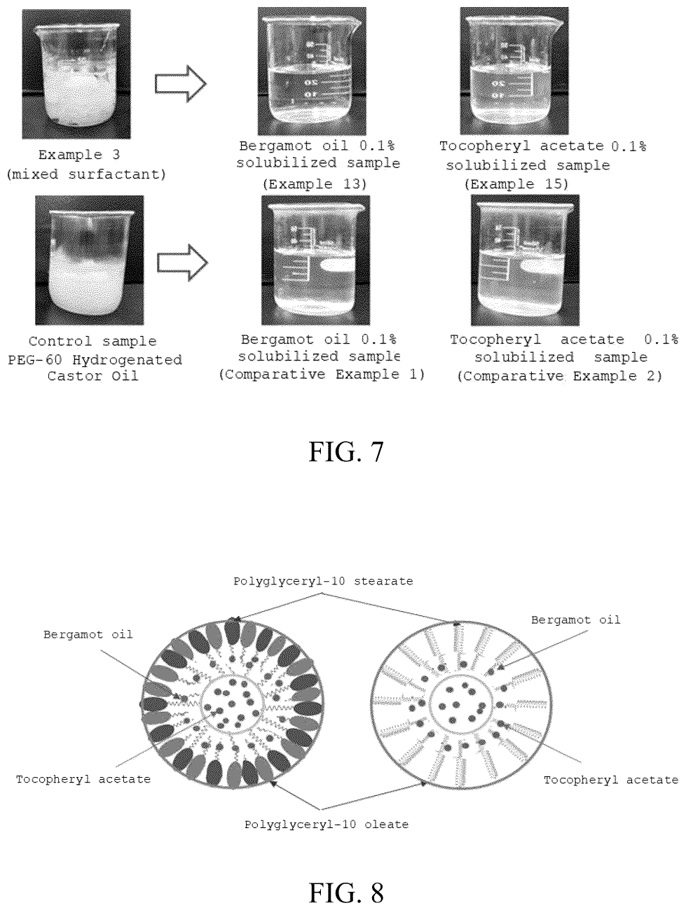

As a result, as shown in FIG. 7, PEG-60 hydrogenated castor oil, which is a conventional surfactant obtained from petroleum, has substantially the same performance as the vegetable mixed surfactant synthesized in the present invention. This means that even if the same amount of vegetable surfactant was used instead of the conventional chemical synthesis raw material, it can exhibit solubilizing power comparable to the conventional one. As the concentration of the surfactant of the present invention increases, micelles are formed, and when bergamot oil and tocopheryl acetate are mixed, the resulting mixture was packed in and adsorbed on micelles to form stable micelles and realize solubilization, as shown in FIG. 8.

Then, when the content of the vegetable surfactant of the present invention (Example 3) was increased, the solubilization power was measured. The ability of an increased amount of the vegetable surfactant of the present invention to solubilize tocopheryl acetate (0.1% by weight) as an oily phase was determined, and was compared with the solubilizing power of PEG-60 hydrogenated castor oil (Table 4).

TABLE 4

| | | | | Com- | Com- | Com- |
|---|---|---|---|---|---|---|
| | | | | par- | par- | par- |
| | | | | ative | ative | ative |
| | Exam- | Exam- | Exam- | Exam- | Exam- | Exam- |
| | ple | ple | ple | ple | ple | ple |
| Component | 17 | 18 | 19 | 3 | 4 | 5 |
| name | (wt %) | (wt %) | (wt %) | (wt %) | (wt %) | (wt %) |
| Example 3 | 1 | 1.2 | 1.5 | — | — | — |
| (vegetable | | | | | | |
| surfactant) | | | | | | |
| PEG-60 | — | — | — | 1 | 1.2 | 1.5 |
| hydrogenated | | | | | | |
| castor oil | | | | | | |
| Tocopheryl | 0.1 | 0.1 | 0.1 | 0.1 | 0.1 | 0.1 |
| acetate | | | | | | |
| (oily phase) | | | | | | |
| Butylene | 5 | 5 | 5 | 5 | 5 | 5 |
| glycol | | | | | | |
| Purified water | 93.9 | 93.7 | 93.4 | 93.9 | 93.7 | 93.4 |
| Total | 100 | 100 | 100 | 100 | 100 | 100 |
| Appearance | Trans- | Trans- | Trans- | Trans- | Trans- | Trans- |
| | parent | parent | parent | parent | parent | parent |
| | liquid | liquid | liquid | liquid | liquid | liquid |
| pH | 6.25 | 6.28 | 6.31 | 6.37 | 6.42 | 6.48 |
| gravity | 1.007 | 1.005 | 1.002 | 1.009 | 1.006 | 1.002 |
| Stability | Stable | Stable | Stable | Stable | Stable | Stable |
| (after 4 | | | | | | |
| weeks at | | | | | | |
| 45° C.) | | | | | | |

*Preparation method: 1) The vegetable surfactant of Example 3 or the surfactant of Comparative Example, tocopheryl acetate and butylene glycol were weighed together, followed by heating to 70° C. to ensure that tocopheryl acetate and butylene glycol were well dissolved in the surfactant. 2) Purified water ( aqueous phase) was weighed and the previously prepared product was slowly dropped while stirring slowly at 20 to 50 rpm, and the solubilization state was compared.

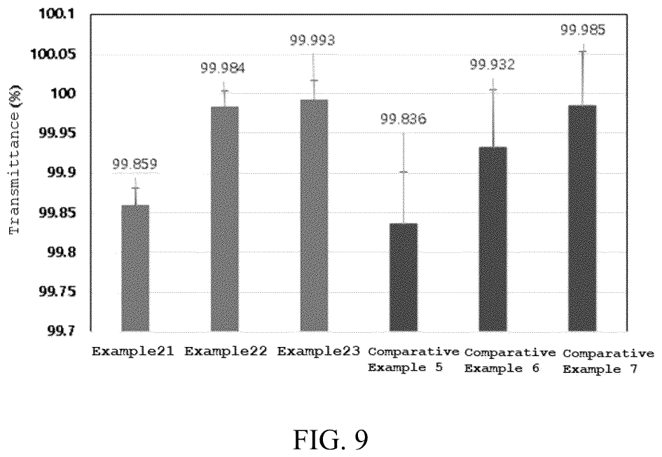

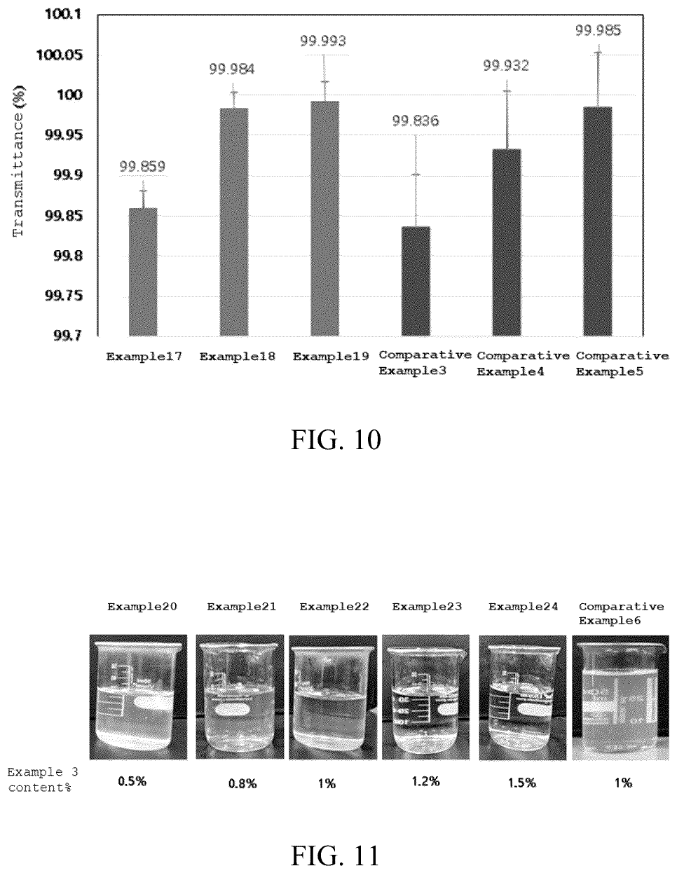

As a result, as can be seen from FIG. 9, PEG-60 hydrogenated castor oil, a conventional surfactant obtained from petroleum, had almost the same performance as the vegetable surfactant synthesized in the present invention. Meanwhile, the result of test of the solubilization power of the vegetable surfactant of the present invention (Example 3) as described above shows that it was difficult to distinguish simply based on appearance. Therefore, for more detailed evaluation, the transmittance was measured using a UV spectrophotometer. The measurement method was as follows. A sample was placed in a quartz cell dedicated to UV absorbance with a width of 1 cm and length of 1 cm and sonicated for 30 seconds to remove air bubbles, and then the transmittance at 890 nm was measured. As a result, as can be seen from FIG. 10, PEG-60 hydrogenated castor oil, a conventional surfactant obtained from petroleum, had almost the same transmittance (transparent liquid) as the vegetable surfactant synthesized in the present invention, which means that the vegetable surfactant had sufficient solubilizing power for the oil component, tocopheryl acetate. As such, it was found that the vegetable surfactant of the present invention has solubilizing power comparable to or greater than that of conventional solubilizing agents.

Next, in order to determine whether or not the solubilizing power of the vegetable surfactant of the present invention (Example 3) is effective under various conditions, whether or not the vegetable surfactant can solubilize lavender oil as an oil phase was determined. When the lavender oil was used at 0.1% by weight and the amount of the vegetable surfactant of the present invention was increased, the solubilization power was evaluated. In all Examples, dipropylene glycol was added (2% by weight) so that the surfactant and lavender oil could be miscible with each other well. In addition, the solubilization power was compared with solubilizing power of PEG-60 hydrogenated castor oil (Table 5)

TABLE 5

| | Ex- | Ex- | Ex- | Ex- | Ex- | Compar- |
|---|---|---|---|---|---|---|
| | am- | am- | am- | am- | am- | ative |
| | ple | ple | ple | ple | ple | Exam- |
| | 20 | 21 | 22 | 23 | 24 | ple |
| Component | (wt | (wt | (wt | (wt | (wt | 6 |
| name | %) | %) | %) | %) | %) | ( wt %) |
| Example 3 | 0.5 | 0.8 | 1 | 1.2 | 1.5 | — |
| (vegetable | | | | | | |
| surfactant) | | | | | | |
| PEG-60 | — | — | — | — | — | 1 |
| hydrogenated | | | | | | |
| castor oil | | | | | | |
| Lavender oil | 0.1 | 0.1 | 0.1 | 0.1 | 0.1 | 0.1 |
| (oily phase) | | | | | | |
| Dipropylene | 2 | 2 | 2 | 2 | 2 | 2 |
| glycol | | | | | | |
| Purified water | 97.4 | 97.1 | 96.9 | 96.7 | 96.4 | 96.9 |
| Total | 100 | 100 | 100 | 100 | 100 | 100 |
| Appearance | Trans- | Trans- | Trans- | Trans- | Trans- | Trans- |
| | lucent | lucent | lucent | lucent | lucent | lucent |
| | liquid | liquid | liquid | liquid | liquid | suspen- |
| | | | | | | sion |
| pH (10% | 6.32 | 6.46 | 6.28 | 6.12 | 6.46 | 6.51 |
| aqueous | | | | | | |
| solution) | | | | | | |
| Gravity | 1.005 | 1.012 | 1.008 | 1.002 | 1.003 | 1.026 |
| Stability | Un- | Un- | Stable | Stable | Stable | Un- |
| (after | stable | stable | | | | stable |
| 4 weeks | | | | | | |
| at 45° C.) | | | | | | |

*Preparation method: 1) The vegetable surfactant of Example 3 or the surfactant of Comparative Example, and lavender oil and dipropylene glycol were weighed together, followed by dissolving while heating to 80° C. 2) The result was cooled to 50° C. or less, and purified water was slowly added thereto while stirring with a disperser at 20 to 100 rpm to compare the solubilization state. 3) After addition was completed, the mixture was cooled to 30° C. to complete preparation.

As a result, as can be seen from FIG. 11, Examples 20 and 21 were translucent, which means that the surfactant could not completely solubilize the lavender oil, whereas Examples 22 to 24 were transparent, which means that complete solubilization appeared. On the other hand, Comparative Example 6 exhibited a translucent suspension, which means that the solubilizing power of Comparative Example 6 was much lower compared to Example 22, having the same composition. As such, it was found that the vegetable surfactant of the present invention has much better solubilizing power than a commonly commercially available solubilizer (PEG-60 hydrogenated castor oil), because hydrogen bonding with water is facilitated and thus the solubilization of lavender oil is better due to a number of hydroxyl (—OH) groups in the hydrophilic moiety of polyglyceryl-10.

### Examples 25 to 28: Preparation of Cosmetic Composition (Emulsion Cream) in Example 3 and Emulsifying Power Test

In this example, a cosmetic composition (emulsion cream) was prepared using the vegetable surfactant of the present invention (Example 3) and the emulsifying power thereof was tested.

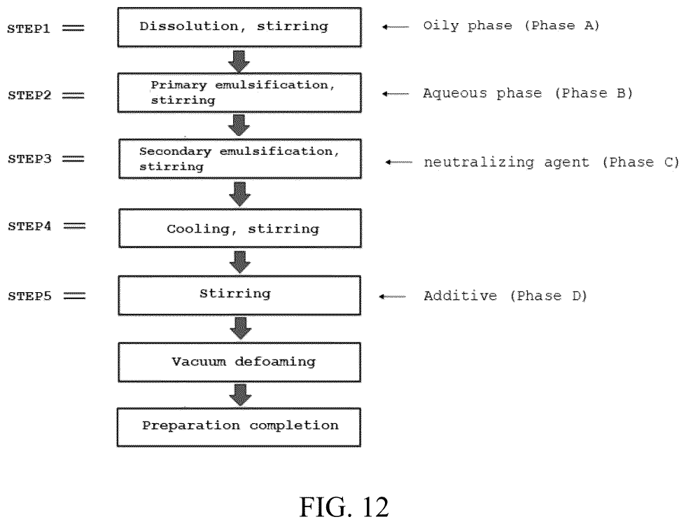

The method of preparing the cosmetic composition includes dissolving the oil phase while stirring (phase A); dissolving the aqueous phase (phase B) while stirring to perform emulsification; neutralizing the resulting product with a neutralizing agent (phase C) while stirring; and cooling and stirring. Meanwhile, when an additive (phase D) was added, the additive was added and stirred at this stage (after cooling and stirring). Finally, after vacuum defoaming, the cosmetic composition (emulsion cream) was completed (FIG. 12). The detailed composition and preparation method are shown in Table 6.

TABLE 6

| | | | | | | Compar- | Compar- |
|---|---|---|---|---|---|---|---|
| | | | | | | ative | ative |
| | | Exam- | Exam- | Exam- | Exam- | Exam- | Exam- |
| | | ple | ple | ple | ple | ple | ple |
| | | 25 | 26 | 27 | 28 | 7 | 8 |
| Component name | | (wt %) | (wt %) | (wt %) | (wt %) | (wt %) | (wt %) |
| Oily | Example 3 | 1 | 1.5 | 1 | 1.5 | — | — |
| phase | (vegetable | | | | | | |
| | surfactant) | | | | | | |
| | PEG-60 | — | — | — | — | 1 | 1 |
| | hydrogenated | | | | | | |
| | castor oil | | | | | | |
| | Bergamot oil | 20 | 30 | — | — | 20 | — |
| | (oily phase) | | | | | | |
| | Tocopheryl | — | — | 20 | 30 | — | 20 |
| | acetate | | | | | | |
| | (oily phase) | | | | | | |
| | Cetearyl alcohol | 5 | 5 | 5 | 5 | 5 | 5 |
| | Butylene glycol | 3 | 3 | 3 | 3 | 3 | 3 |
| Aqueous | Glycerin | 2 | 2 | 2 | 2 | 2 | 2 |
| phase | 2% Carbomer-940 | 15 | 15 | 15 | 15 | 15 | 15 |
| | solution | | | | | | |
| | Purified water | 50.5 | 40 | 50.5 | 40 | 50.5 | 50.5 |
| Neutralizing | 10% potassium | 1.5 | 1.5 | 1.5 | 1.5 | 1.5 | 1.5 |
| agent | hydroxide | | | | | | |
| | solution | | | | | | |
| | Purified water | 2 | 2 | 2 | 2 | 2 | 2 |
| Total | | 100 | 100 | 100 | 100 | 100 | 100 |
| Appearance | | Emul- | Emul- | Emul- | Emul- | Emul- | Emul- |
| | | sion | sion | sion | sion | sion | sion |
| | | cream | cream | cream | cream | cream | cream |

| | | | | | | |
|---|---|---|---|---|---|---|
| pH | 5.82 | 5.86 | 5.93 | 5.91 | 5.91 | 5.97 |
| Specific gravity | 0.997 | 0.992 | 0.996 | 0.991 | 0.996 | 0.997 |
| Stability (after 4 | Stable | Stable | Stable | Stable | Stable | Stable |
| weeks at 45° C.) | | | | | | |

*Preparation method: 1) oily phase and aqueous phase were heated to 80° C., followed by dissolving. 2) aqueous phase was mixed with oily phase, followed by stirring at 4,000 rpm for 5 minutes with a homogenizer to obtain emulsion. 3) a neutralizing agent was added thereto, followed by stirring at 4,000 rpm for 3 minutes to increase the viscosity. 4) The result was cooled to 30° C., followed by vacuum degassing to complete preparation.

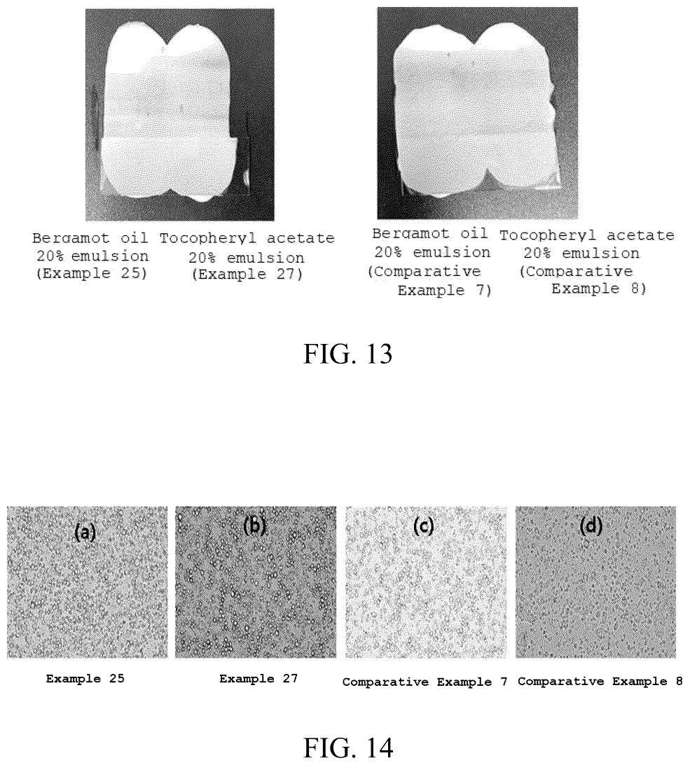

As a result, as can be seen from FIG. 13, the vegetable surfactants synthesized in the present invention (Examples 25 and 27) provided denser and finer emulsion particles and was shinier than PEG-60 hydrogenated castor oil (Comparative Examples 7 and 8), which is a conventional surfactant obtained from petroleum. These results show that Examples and 27 had better surfactant than conventional surfactants.

The emulsified particles of the emulsion cream prepared above were observed under a microscope. FIG. 14(a) is an image showing the emulsion particle size of Example 25 emulsified with 20% bergamot oil, FIG. 14(b) is an image showing the emulsion particle size of Example 27 emulsified with 20% tocopheryl acetate, and FIG. 14(c) is an image showing the emulsion particle size of Comparative Example 25 and 27 emulsified with PEG-60 hydrogenated castor oil, measured under the same conditions. Examples 25 and 27 show a uniform and dense distribution of emulsion particle sizes, while Comparative Example 7 shows a mixture of large and small emulsion particle sizes, and Comparative Example 8 exhibits some particle aggregation due to slightly poor emulsification power. This indicates that the vegetable surfactant of the present invention had better emulsifying power than the conventional surfactant.

### Examples 29 to 33: Preparation of Cosmetic Composition (Anti-Aging Cream) Using Example 3

In this example, cosmetic compositions (anti-aging creams) were prepared using the vegetable surfactant of the present invention (Example 3). The detailed composition and preparation method are shown in Table 7.

TABLE 7

| | | | | | | | Compar- | Compar- |
|---|---|---|---|---|---|---|---|---|
| | | | | | | | ative | ative |
| | | Exam- | Exam- | Exam- | Exam- | Exam- | Exam- | Exam- |
| | | ple | ple | ple | ple | ple | ple | ple |
| | | 29 | 30 | 31 | 32 | 33 | 9 | 10 |
| Component name | | (wt %) | (wt %) | (wt %) | (wt %) | (wt %) | (wt %) | (wt %) |
| Oily | Example 3 | 2 | 2 | 2 | 2 | 2 | — | — |
| phase | (vegetable surfactant) | | | | | | | |
| | PEG-60 | — | — | — | — | — | 2 | 2 |
| | hydrogenated | | | | | | | |
| | castor oil | | | | | | | |
| | Apricot oil | 10 | 10 | 10 | 10 | 10 | 10 | 10 |
| | Glyceryl | 5 | 5 | 5 | 5 | 5 | 5 | 5 |
| | monostearate | | | | | | | |
| | Stearic acid | 0.5 | 0.5 | 0.5 | 0.5 | 0.5 | 0.5 | 0.5 |
| | Cetearyl | 1.5 | 1.5 | 1.5 | 1.5 | 1.5 | 1.5 | 1.5 |
| | alcohol | | | | | | | |
| Aqueous | Dipropylene | 5 | 5 | 5 | 5 | 5 | 5 | 5 |
| phase | glycol | | | | | | | |
| | Glycerin | 2 | 2 | 2 | 2 | 2 | 2 | 2 |
| | Butylene | 3 | 3 | 3 | 3 | 3 | 3 | 3 |
| | glycol | | | | | | | |
| | EDTA-2Na | 0.03 | 0.03 | 0.03 | 0.03 | 0.03 | 0.03 | 0.03 |
| | 1,2- | 2 | 2 | 2 | 2 | 2 | 2 | 2 |
| | hexanediol | | | | | | | |
| | Sodium | 0.05 | 0.05 | 0.05 | 0.05 | 0.05 | 0.05 | 0.05 |
| | citrate | | | | | | | |
| | 2% Carbomer- | 20 | 20 | 20 | 20 | 20 | 20 | 20 |
| | 940 solution | | | | | | | |
| | Purified | 40.12 | 40.02 | 40.02 | 35.12 | 39.02 | 40.02 | 40.02 |
| | water | | | | | | | |
| Neutralizing | 10% | 1.8 | 1.8 | 1.8 | 1.8 | 1.8 | 1.8 | 1.8 |
| agent | potassium | | | | | | | |
| | hydroxide | | | | | | | |
| | solution | | | | | | | |
| | Purified | 2 | 2 | 2 | 2 | 2 | 2 | 2 |
| | water | | | | | | | |
| Additives | Acetyl | — | 0.1 | — | — | 0.05 | 0.1 | — |
| | hexapeptide- | | | | | | | |
| | 8 | | | | | | | |
| | AHK-Cu | — | — | 0.1 | — | 0.05 | — | 0.1 |
| | Oxygen water | — | — | — | 5 | 1 | — | — |
| | (30 ppm) | | | | | | | |

| | | | | | | | |
|---|---|---|---|---|---|---|---|
| Total | 100 | 100 | 100 | 100 | 100 | 100 | 100 |
| Appearance | Emul- | Emul- | Emul- | Emul- | Emul- | Emul- | Emul- |
| | sion | sion | sion | sion | sion | sion | sion |
| | cream | cream | cream | cream | cream | cream | cream |
| Stability (after 4 | Stable | Stable | Stable | Stable | Stable | Stable | Stable |
| weeks at 45° C.) | | | | | | | |
| pH | 6.11 | 6.15 | 6.21 | 6.18 | 6.13 | 6.08 | 6.05 |
| Viscosity | 35,600 | 35,800 | 35,200 | 35,200 | 36,100 | 34,600 | 34,800 |
| (spindle 3, 12 rpm, 1 | | | | | | | |
| min, cps) | | | | | | | |
| Gravity | 0.997 | 0.997 | 0.997 | 0.997 | 0.997 | 0.997 | 0.997 |

*Preparation method: 1) oily phase and aqueous phase were heated to 80° C., followed by dissolving. 2) aqueous phase was mixed with oily phase, followed by stirring at 4,000 rpm for 5 minutes with a homogenizer to obtain emulsion. 3) a neutralizing agent was added thereto, followed by stirring at 4,000 rpm for 3 minutes to increase the viscosity. 4) The result was cooled to 45° C., followed by stirring at 4,000 rpm for 3 minutes to increase the viscosity. 4) The result was cooled to 45° C., and additives were added thereto and stirred at 4,000 rpm for 2 minutes to obtain a homogeneous dispersion. 5) the result was cooled to 30° C., followed by vacuum degassing to complete preparation.

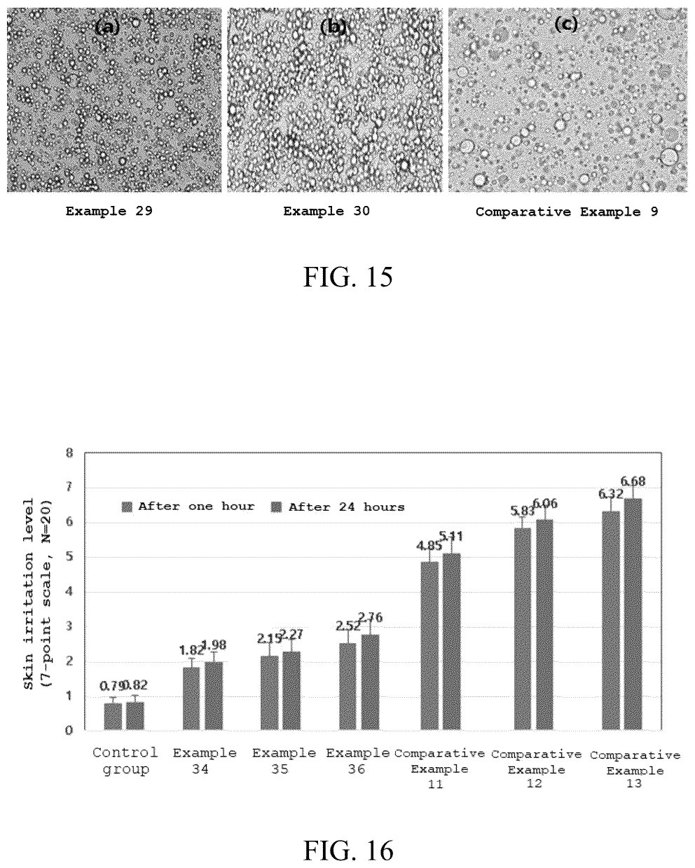

The results of observation of the emulsified particles of the anti-aging cream prepared above under a microscope were shown in FIG. 15, wherein (a) shows a placebo sample containing no additives while using 2% of the vegetable surfactant of the present invention, (b) shows an emulsion cream containing 0.1% (1,000 ppm) of acetylhexapeptide, an effective ingredient, while using 2% of the vegetable surfactant of the present invention, and (c) is an emulsion cream containing the same amount of a typical general surfactant, PEG-60 hydrogenated castor oil and the same effective ingredients as Example 30. This shows that the vegetable surfactants synthesized in the present invention (Examples 29 and 30) have finer emulsifying particle size and thus excellent emulsifying power compared to Comparative Example 9.

### Experimental Example 1: Test of Various Skin Functionalities of the Vegetable Surfactant of the Present Invention

In this experimental example, various skin functionalities were tested using the vegetable surfactant of the present invention.

### 1) Skin Irritation Test

To evaluate skin safety, skin irritation was compared between the vegetable surfactant of the present invention and conventional surfactants. For this purpose, skin irritation was evaluated through a patch test, and the details are as follows: men and women (20 people) were selected, a 7-point scale was used, and the patch was applied to the lower arms of both forearms for 24 hours. After patch removal and washing with purified water, the results of evaluating skin sensitization after 1 hour and skin condition after 24 hours are shown in brief. The result of the skin irritation test of the control group, Examples 34 to 36, and Comparative Examples 11 to 13, as shown in FIG. 16, showed that each sample was used at a concentration 30 times higher than the commonly used concentration to obtain more realistic results within a short time. The control group was a sample of the base itself containing no surfactant and thus caused almost no irritation. However, in Comparative Examples 11 to 13, red erythema appeared on the skin after 1 hour and 24 hours, and the degree of skin irritation ranged from 4.85 to 6.68 which is higher than a normal level. On the other hand, Examples 34 to 36 had low irritation of 1.82 to 2.76. The surfactant of the present invention, developed only from a vegetable ingredient, is non-irritating because it has a hydrophilic moiety containing the moisturizing component of polyglyceryl-10 and a lipophilic moiety based on combination of oleic acid and stearic acid to provide softening effect. However, in Comparative Examples, the oil phase of hydrogenated castor oil was combined with the hydrophilic moiety of polyethylene glycol 60 mol, and some of the functional groups caused skin irritation.

TABLE 8

| | | | | | | Com- | Com- | Com- |
|---|---|---|---|---|---|---|---|---|
| | | | Ex- | Ex- | Ex- | par- | par- | par- |
| | | | am- | am- | am- | ative | ative | ative |
| | | Con- | ple | ple | ple | Exam- | Exam- | Exam- |
| | | trol | 34 | 35 | 36 | ple | ple | ple |
| Component | | group | (wt | (wt | (wt | 11 | 12 | 13 |
| name | | (wt %) | %) | %) | %) | (wt %) | (wt %) | ( wt %) |
| 1 | Example 3 | — | 30 | 40 | 50 | — | — | — |
| | (Vegetable | | | | | | | |
| | surfactant) | | | | | | | |
| 2 | PEG-60 | — | — | — | — | 30 | 40 | 50 |
| | hydrogenated | | | | | | | |
| | castor | | | | | | | |
| | oil | | | | | | | |
| 3 | Glycerin | 30 | 30 | 30 | 30 | 30 | 30 | 30 |
| 4 | Purified | 70 | 40 | 30 | 20 | 40 | 30 | 20 |
| | water | | | | | | | |

| | | | | | | | |
|---|---|---|---|---|---|---|---|
| Total | 100 | 100 | 100 | 100 | 100 | 100 | 100 |

*Preparation method: 1) 1, 2, and 3 were weighed, heated to 70° C., dissolved, and mixed with a spoon for 3 minutes. 2) 4 was added thereto, stirred at 3,000 rpm for 5 minutes, cooled to 30° C., and then vacuum degassed.

### 2) Cytotoxicity Evaluation

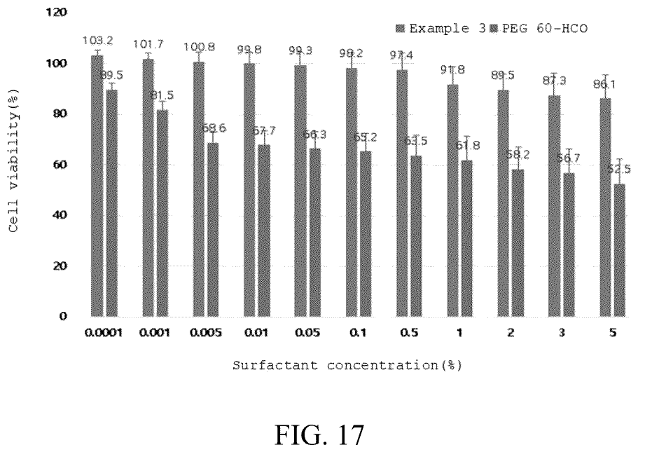

Cytotoxicity was evaluated to further verify skin stability. The cytotoxicity tests are used as an indicator to evaluate skin irritation and were performed using conventional surfactants. When the content of the vegetable surfactant of the present invention (Example 3) and PEG-60 hydrogenated castor oil (PEG 60-HCO), a typical general surfactant, were increased in the order of 0.0001%, 0.001%, 0.005%, 0.1%, and 0.5%, the cell viability was tested. The result, as shown in FIG. 17, shows that, in Example 3, the cell viability was 103.2% to 86.1%, which indicates that there was no significant effect and there was almost no cytotoxicity, whereas the cell viability of PEG 60-hydrogenated castor oil was 89.5% at a low concentration and was significantly decreased to 52.5% as the concentration increased. These results showed that the vegetable surfactant of the present invention was less irritating than the conventional PEG-60 hydrogenated castor oil.

### 3) Evaluation of Skin Moisturizing Power

As can be seen from above, the vegetable surfactant of the present invention has excellent solubilizing power and can thus be used as a solubilizing agent. Meanwhile, the skin moisturizing power was evaluated to determine whether or not it was a raw material having excellent moisturizing power. For this purpose, both short-term moisturizing power and long-term moisturizing power were evaluated.

First, the short-term moisturizing power evaluation was conducted on 20 subjects (20s to 60s, men and women), the measurements was performed three times and the average thereof was expressed. A predetermined amount of 2 g of each sample was applied to the face twice a day (morning and evening) for 4 weeks, and the moisture content present in the skin was measured using a moisture meter in an incubator unaffected by humidity at the same spot. The amount of moisture present in the skin from application to 8 hours later was measured. The test samples were non-coated, placebo, Example 37, and Comparative Example 14 in Table 9, and the test device used herein was an Aramo TS (device for measuring moisturizing power) equipped with a software program.

TABLE 9

| | | | | Comparative |
|---|---|---|---|---|
| | | Placebo 1 | Example | Example 14 |
| Component name | | (wt %) | 37 (wt %) | (wt %) |
| Oily phase | Example 3 (vegetable surfactant) | — | 5 | — |
| | PEG-60 hydrogenated castor oil | — | — | 5 |
| | Mineral oil | 10 | 10 | 10 |
| | Cetearyl alcohol | 5 | 5 | 5 |
| Aqueous phase | EDTA-2Na | 0.03 | 0.03 | 0.03 |
| | 1,2-hexanediol | 2 | 2 | 2 |
| | Sodium citrate | 0.05 | 0.05 | 0.05 |
| | 2% Carbomer-940 solution | 20 | 20 | 20 |
| | Purified water | 56.92 | 51.92 | 51.92 |
| Neutralizing agent | 10% potassium hydroxide solution | 4 | 4 | 4 |
| | Purified water | 2 | 2 | 2 |

| | | | |
|---|---|---|---|
| Total | 100 | 100 | 100 |
| Appearance | emulsion cream | emulsion cream | emulsion cream |
| pH | 6.67 | 6.68 | 6.62 |
| Viscosity (spindle 3, 12 rpm, 1 min, cps) | 32,670 | 33,760 | 33,750 |
| Specific gravity | 0.998 | 0.996 | 0.997 |

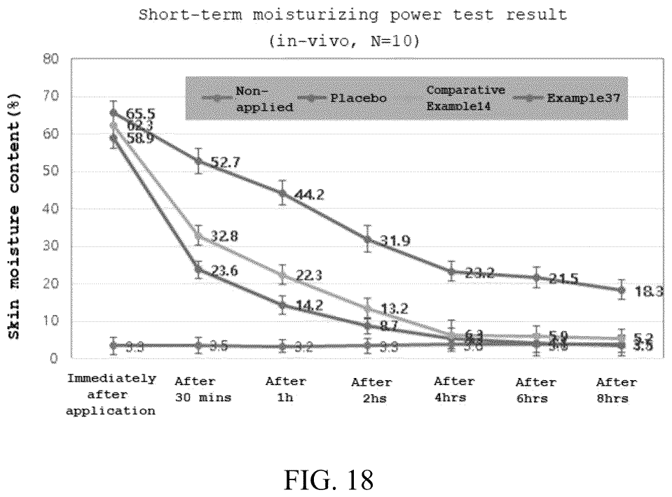

As a result, as shown in FIG. 18, the moisture content of the skin immediately after application ranged from 58.9% to 65.5%, and the moisture content in the skin decreased over time. The non-applied sample group had a moisture content of 3.3% to 3.6%, without no significant change, and the placebo sample had a moisture content of 58.9% due to the presence of moisture immediately after application, but, after 8 hours, the moisture content was decreased to 3.5%, which is similar to the non-applied sample. Meanwhile, in Comparative Example 14, the moisture content was 62.3% immediately after application, but decreased to 13.2% after 2 hours, to 6.3% after 4 hours, and to 5.2% after 8 hours. This moisture content had no significant difference from 3.7% which is the moisture content of the non-applied sample group.

However, in Example 37, the moisture content was 65.5% immediately after application, but gradually decreased to 31.9% after 2 hours, 23.2% after 4 hours, 21.5% after 6 hours, and 18.3% after 8 hours, which were significantly higher than that of the placebo and Comparative Example 14. When the moisture content is converted to the ratio (number), the moisture content increased by 5.2 times compared to the placebo after 8 hours and increased by 3.5 times compared to Comparative Example 14. The moisture content is increased because the hydrophilic moiety of the vegetable surfactant (Example 3) synthesized in the present invention contains a large amount of hydroxyl groups (—OH), which forms a hydrogen bond with moisture (H₂O) present in the skin. This shows that the vegetable surfactant of the present invention had excellent short-term moisturizing ability.

As described above, the short-term moisturizing power was excellent. A skin moisturizing power was evaluated after application for a long period of time (4 weeks) to determine whether or not it had moisturizing power even for a long period of time. The long-term moisturizing power evaluation was conducted on 20 subjects (20s to 60s, men and women), measurement was performed three times and the average thereof was shown. A predetermined amount of 2 g of each sample was applied to the face twice a day (morning and evening) for 4 weeks, and the moisture content in the skin was measured using a moisture meter in an incubator unaffected by humidity at the same spot. The amount of moisture was measured before application and from 1 week to 4 weeks after application, and finally, the measurement results of the moisturizing improvement effect were graphed. The test samples were non-coated, placebo, Example 37, and Comparative Example 14 in Table 9, and the test device used herein was Aramo TS (device for measuring moisturizing power) equipped with a software program.

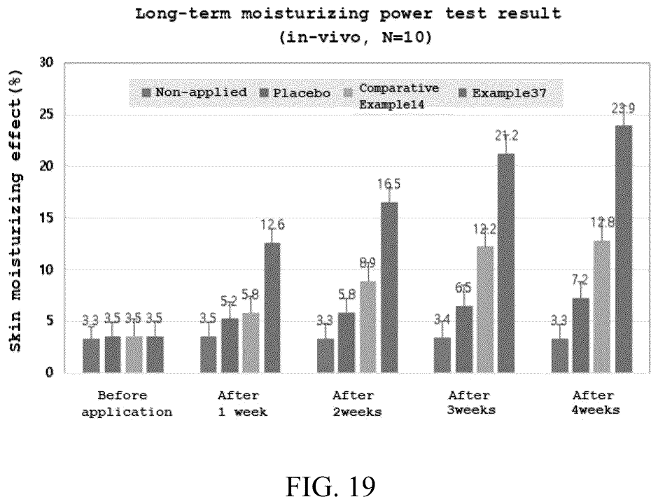

As a result, as shown in FIG. 19, the non-application group had a low moisture content of 3.3% to 3.5%, and the placebo sample had a moisturizing effect of 5.2% after 1 week and 7.2% after 4 weeks. Meanwhile, in Comparative Example 14, the moisturizing effect increased to 5.8% after 1 week and 12.8% after 4 weeks. However, in Example 37, the moisturizing power was increased by 12.6% after 1 week, 16.5% after 2 weeks, 21.2% after 3 weeks, and 23.9% after 4 weeks. More specifically, the moisturizing power after 1 week to 3 weeks increased slightly, but the moisturizing power was increased by 1.87 times after 4 weeks. This increase in moisturizing power is because the hydrophilic moiety of the vegetable surfactant (Example 3) synthesized in the present invention contains a great amount of hydroxyl groups (—OH), has excellent adhesion to the skin, maintains skin moisture for a long time, and forms hydrogen bonds with purified water. This indicates that the vegetable surfactant of the present invention has excellent long-term moisturizing power.

### 4) Skin Whitening Evaluation

A clinical test to determine whitening effect was performed on the surfactant prepared using 2% by weight of niacinamide, known as a typical whitening ingredient (Example 38). Placebo is a base material that is not related to the whitening effect, Comparative Example 15 is a formulation using PEG-60 hydrogenated castor oil, a typical general surfactant, and Example 38 is a formulation using 5% by weight of the vegetable surfactant of the present invention (Example 3) and 2% by weight of niacinamide. In-vivo clinical evaluation was conducted under the same prescription conditions.

TABLE 10

| | | | | Comparative |
|---|---|---|---|---|
| | | Placebo | Example | Example 15 |
| Component name | | (wt %) | 38 (wt %) | (wt %) |
| Oily phase | Example 3 (vegetable surfactant) | — | 5 | — |
| | PEG-60 hydrogenated castor oil | — | — | 5 |
| | Mineral oil | 10 | 10 | 10 |
| | Cetearyl alcohol | 5 | 5 | 5 |
| Aqueous phase | EDTA-2Na | 0.03 | 0.03 | 0.03 |
| | 1,2-hexanediol | 2 | 2 | 2 |
| | Sodium citrate | 0.05 | 0.05 | 0.05 |
| | 2% Carbomer-940 solution | 20 | 20 | 20 |
| | Purified water | 56.92 | 44.92 | 44.92 |
| Neutralizing agent | 10% potassium hydroxide solution | 4 | 4 | 4 |
| | Purified water | 2 | 2 | 2 |
| Additives | Niacinamide | — | 2 | 2 |
| | Purified water | — | 5 | 5 |

| | | | |
|---|---|---|---|
| Total | 100 | 100 | 100 |
| Appearance | Emulsion cream | Emulsion cream | Emulsion cream |
| pH | 6.67 | 6.68 | 6.62 |
| Viscosity (spindle 3, 12 rpm, 1 min, cps) | 32,670 | 33,760 | 33,750 |
| Specific gravity | 0.998 | 0.996 | 0.997 |

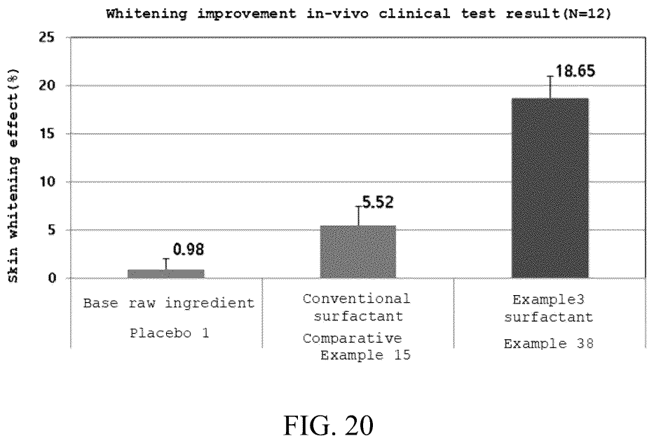

As a result, as shown in FIG. 20, in Placebo 1, the whitening effect after 4 weeks was improved by 0.98% compared to before application, showing almost no effect, and in Comparative Example 15, the whitening effect was improved by 5.52% compared to before application. Example 38 had a whitening effect of 18.65%. These results showed that Example 38 had a whitening effect that was 3.38 times higher than Comparative Example 15 and was 19.03 times higher than Placebo 1. These results are due to the fact that niacinamide, a whitening ingredient, has a lot of hydroxyl (—OH) groups of polyglyceryl-10 bonded to the hydrophilic group of the vegetable surfactant of Example 38, to facilitate hydrogen bonds with purified water (H₂O). This synergetic effect results in better whitening effect.

### 5) Evaluation of Fine Wrinkle Amelioration

The surfactant was prepared by further adding the active ingredient acetylhexapeptide-8 (Example 39), and a clinical test (N=12, men and women in their 20s to 60s) to determine the effect of improving fine wrinkles on the skin was conducted on Example 39, Placebo and Comparative Examples. The test method used herein was a skin wrinkle amelioration meter from Aramo TS (Korea). A predetermined amount of each sample was applied to the entire face, 2 g, twice a day (morning and evening) for 4 weeks, and the amelioration of fine wrinkles after 4 weeks was measured numerically in the same spot around the eyes (around the eyebrows, where fine wrinkles are most common). 12 subjects were selected for each sample, the measurement was performed three times, and the average thereof was used to quantify the final evaluation results.

TABLE 11

| | | | | Comparative |
|---|---|---|---|---|
| | | Placebo 2 | Example 39 | Example 16 |
| Component name | | (wt %) | (wt %) | (wt %) |
| Oily phase | Example 3 (vegetable surfactant) | — | 5 | — |
| | PEG-60 hydrogenated castor oil | — | — | 5 |
| | mineral oil | 10 | 10 | 10 |
| | Cetearyl alcohol | 5 | 5 | 5 |
| Aqueous phase | EDTA-2Na | 0.03 | 0.03 | 0.03 |
| | 1,2-hexanediol | 2 | 2 | 2 |
| | Sodium citrate | 0.05 | 0.05 | 0.05 |
| | 2% Carbomer-940 solution | 20 | 20 | 20 |
| | Purified water | 56.92 | 48.82 | 48.82 |
| Neutralizing agent | 10% potassium hydroxide solution | 4 | 4 | 4 |
| | Purified water | 2 | 2 | 2 |
| Additives | Acetyl hexapeptide-8 | — | 0.1 | 0.1 |
| | Purified water | — | 3 | 3 |

| | | | |
|---|---|---|---|
| Total | 100 | 100 | 100 |
| Appearance | Emulsion cream | Emulsion cream | Emulsion cream |
| pH | 6.32 | 6.18 | 6.12 |
| Viscosity (spindle 3, 12 rpm, 1 min, cps) | 32,670 | 33,760 | 33,750 |
| Specific gravity | 0.998 | 0.996 | 0.997 |

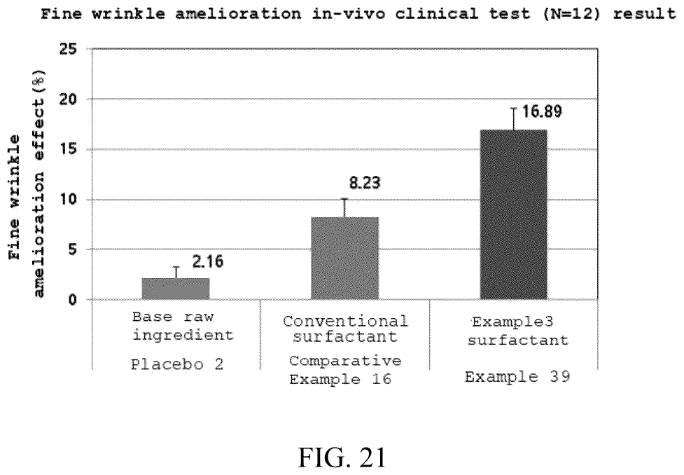

As a result, as can be seen from FIG. 21, Placebo 2 had almost no effect of ameliorating fine wrinkles after 4 weeks, with an amelioration of 2.16% compared to before application, Comparative Example 16 had a fine wrinkle amelioration of 8.23% compared to before application, and Example 39 had a fine wrinkle amelioration of 16.89%. These results showed that Example 39 had a fine wrinkle amelioration effect that corresponded to 2.05 times higher than Comparative Example 16 and corresponded to 7.8 times higher than Placebo 2. These results show that acetylhexapeptide-8 as a raw material is effective in ameliorating fine wrinkles, and the hydrophilic moiety of the vegetable surfactant of the present invention is combined with a large number of —OH groups of polyglyceryl-10, to provide excellent moisturizing power, and a better fine wrinkle effect was obtained due to the synergetic effect.

### 6) Evaluation of Skin Lifting Improvement

An in-vivo (N=12) experiment to determine skin lifting improvement effect was performed using Placebo 3, Comparative Example 17, and Example 40. The skin lifting test used herein was a skin elasticity meter from Aramo TS (Korea). A predetermined amount of each sample was applied to the entire face twice a day (morning and evening) for 4 weeks, and skin lifting was measured on the selected same spot after 4 weeks. For each sample, 12 subjects were selected, measurement was performed three times, and the average thereof was applied to quantify the final results.

TABLE 12

| | | | | Comparative |
|---|---|---|---|---|
| | | Placebo 3 | Example 40 | Example 17 |
| Component name | | (wt %) | (wt %) | (wt %) |
| Oily phase | Example 3 (vegetable surfactant) | — | 5 | — |
| | PEG-60 hydrogenated castor oil | — | — | 5 |
| | Mineral oil | 10 | 10 | 10 |
| | Cetearyl alcohol | 5 | 5 | 5 |
| Aqueous phase | EDTA-2Na | 0.03 | 0.03 | 0.03 |
| | 1,2-hexanediol | 2 | 2 | 2 |
| | Sodium citrate | 0.05 | 0.05 | 0.05 |
| | 2% Carbomer-940 solution | 20 | 20 | 20 |
| | Purified water | 51.92 | 44.42 | 44.42 |
| Neutralizing agent | 10% potassium hydroxide solution | 4 | 4 | 4 |
| | Purified water | 2 | 2 | 2 |
| Additives | Retinol (100,000 IU/g) | — | 2.5 | 2.5 |
| | Purified water | 5 | 5 | 5 |

| | | | |
|---|---|---|---|
| Total | 100 | 100 | 100 |
| Appearance | Emulsion cream | Emulsion cream | Emulsion cream |
| pH | 6.32 | 6.18 | 6.12 |
| Viscosity (spindle 3, 12 rpm, 1 min, cps) | 35,200 | 36,300 | 36,700 |
| Specific gravity | 0.998 | 0.997 | 0.999 |

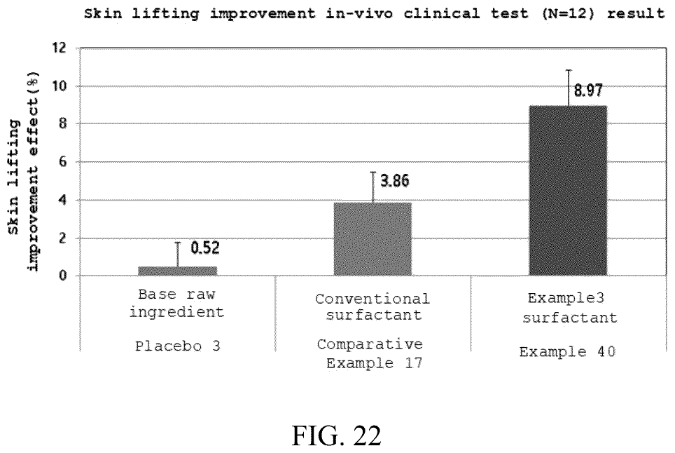

As a result, as can be seen from FIG. 22, Placebo 3 showed a very slight lifting increase of about 0.52% compared to before application, and Comparative Example 17 showed a significant skin lifting improvement of 3.86% compared to Placebo 3. Meanwhile, Example 40 showed a significantly improved skin lifting of 8.97%. These results showed that Example 40 had skin lifting improvement effect that was 2.3 times higher than Comparative Example 17 and was 17.25 times higher than Placebo 3.

As can be seen above, the vegetable surfactant of the present invention exhibits excellent skin stability, very low cytotoxicity, excellent short-term moisturizing effect and long-term moisturizing effect, and superior skin improvement effects such as fine wrinkle amelioration, skin whitening and skin lifting improvement effects. The vegetable surfactant is applicable to various cosmetic compositions and various formulations of cosmetic compositions containing the vegetable surfactant of the present invention were prepared.

### Examples 41 to 43: Preparation of Cosmetic Composition (Cleansing Oil) Containing Vegetable Surfactant of the Present Invention

In this example, a cosmetic composition containing the vegetable surfactant of the present invention (Example 3) was prepared. For example, cleansing oil was prepared and the detailed composition thereof is shown in Table 13 below.

TABLE 13

| | | Example | Example | Example |
|---|---|---|---|---|
| Component name | | 41 (wt %) | 42 (wt %) | 43 (wt %) |
| Phase A | Example 3 | 15 | 20 | 25 |
| | (vegetable | | | |
| | surfactant) | | | |
| Phase B | Cetyl | 30 | 25 | 20 |
| | ethylhexanoate | | | |
| | Ethylhexyl | 20 | 15 | 18 |
| | palmitate | | | |
| | Jojoba oil | 10 | 12 | 10 |
| | Apricot oil | 8 | 10 | 12 |
| | Capric/caprylic | 6.85 | 6.95 | 6.95 |
| | triglyceride | | | |
| | Argan tree oil | 5 | 5.9 | 2.9 |
| | Tocopheryl | 0.1 | 0.1 | 0.1 |
| | acetate | | | |
| | Ascorbyl tetra- | 3 | 3 | 3 |
| | isopalmitate | | | |
| | Lavender oil | 0.05 | 0.05 | 0.05 |
| Phase C | Purified water | 1.7 | 1.7 | 1.7 |
| | Purslane | 0.1 | 0.1 | 0.1 |
| | extract | | | |
| | Green tea | 0.1 | 0.1 | 0.1 |
| | extract | | | |
| | Hibiscus | 0.1 | 0.1 | 0.1 |
| | extract | | | |

| | | | |
|---|---|---|---|
| Total | 100 | 100 | 100 |
| Appearance | Transparent | Transparent | Transparent |
| | liquid | liquid | liquid |
| pH | 5.71 | 5.76 | 5.72 |
| Odor | Lavender | Lavender | Lavender |
| | scent | scent | scent |
| Specific Gravity | 0.985 | 0.987 | 0.977 |
| Stability (after 4 | Stable | Stable | Stable |
| weeks at 45° C.) | | | |

*Preparation method: 1) Phase A and Phase B were weighed, followed by dissolution while heating to 75 to 80° C. 2) Phase C was stirred along with the result at 120 to 500 rpm for 5 minutes. 3) When a transparent microemulsion was obtained, it was further stirred at 120 to 500 rpm for 5 minutes. 4) The result was cooled to 30° C., followed by vacuum defoaming to complete preparation.

### Examples 44 to 46: Preparation of Cosmetic Composition (Skin Toner) Containing Vegetable Surfactant of the Present Invention

In this example, a cosmetic composition containing the vegetable surfactant of the present invention (Example 3) was prepared. For example, skin toner was prepared and the detailed composition thereof is shown in Table 14 below.

TABLE 14

| | | Example | Example | Example |
|---|---|---|---|---|
| Component name | | 44 (wt %) | 45 (wt %) | 46 (wt %) |
| Phase A | Glycerin | 3 | 4 | 5 |
| | Dipropylene | 8 | 5 | 3 |
| | glycol | | | |
| | Ethyl | 0.05 | 0.05 | 0.05 |
| | hexylglycerin | | | |
| | Purified water | 2 | 3 | 5 |
| Phase C | 1,3-butylene | 5 | 5 | 5 |
| | glycol | | | |
| | 1,2-hexanediol | 2 | 2 | 2 |
| | EDTA-2Na | 0.03 | 0.03 | 0.03 |
| | Allantoin | 0.01 | 0.01 | 0.01 |
| | Ethyl ascorbic | 0.2 | 0.2 | 0.2 |
| | acid | | | |
| | Acetyl | 0.05 | 0.05 | 0.05 |
| | hexapeptide-8 | | | |
| | Purified water | 79.41 | 80.26 | 79.10 |
| | Peppermint oil | 0.03 | 0.03 | 0.03 |
| | Example 3 | 0.1 | 0.2 | 0.3 |
| | (vegetable | | | |
| | surfactant) | | | |
| Phase D | Sodium | 0.01 | 0.02 | 0.03 |
| | hyaluronate | | | |
| | Idebenone | 0.01 | 0.05 | 0.1 |
| | Red ginseng | 0.1 | 0.1 | 0.1 |
| | extract | | | |

| | | | |
|---|---|---|---|
| Total | 100 | 100 | 100 |
| Appearance | Transparent | Transparent | Transparent |
| | liquid | liquid | liquid |
| pH | 5.73 | 5.82 | 5.87 |
| Specific gravity | 0.998 | 0.996 | 0.995 |
| Stability (3 months | Stable | Stable | Stable |
| at 45° C.) | | | |

*Preparation method: 1) Phase A was weighed and dissolved while heating to 55 to 60° C. 2) The result was mixed with Phase B, followed by stirring with a disperser. 3) Phase C was further added thereto and dissolved while stirring. 4) The result was cooled to 30° C., followed by vacuum defoaming to complete preparation.

### Examples 47 to 49: Preparation of Cosmetic Composition (Emulsion Lotion) Containing Vegetable Surfactant of the Present Invention

In this example, a cosmetic composition containing the vegetable surfactant of the present invention (Example 3) was prepared. For example, emulsion lotion was prepared and the detailed composition thereof is shown in Table 15 below.

TABLE 15

| | | Example | Example | Example |
|---|---|---|---|---|
| Component name | | 47 (wt %) | 48 (wt %) | 49 (wt %) |
| Phase A | Example 3 | 3 | 5 | 10 |
| (oily | (vegetable | | | |
| phase) | surfactant) | | | |
| | Cetostearyl | 1.5 | 1.5 | 1.5 |
| | alcohol | | | |
| | Behenyl alcohol | 1 | 1 | 1 |
| | Stearic acid | 0.5 | 0.5 | 0.5 |
| | Glyceryl | 0.5 | 0.5 | 0.5 |
| | stearate | | | |
| | Cetyl | 3 | 5 | 7 |
| | ethylhexanoate | | | |
| | Macadamia oil | 0.8 | 1 | 2 |
| | Argan oil | 0.5 | 0.5 | 1 |
| | Olive oil | 0.2 | 0.2 | 0.2 |
| | Tocopheryl | 0.1 | 0.1 | 0.1 |
| | acetate | | | |
| | Camellia oil | 0.1 | 0.2 | 0.3 |
| Phase B | Carbomer-941 | 0.2 | 0.3 | 0.4 |
| (aqueous | Transparent | 0.1 | 0.05 | 0.02 |
| phase 1) | xanthan gum | | | |
| | Purified water | 20 | 20 | 20 |
| Phase C | 1,3-Butylene | 3 | 5 | 7 |
| (Aqueous | glycol | | | |
| phase 2) | Glycerin | 5 | 3 | 2 |
| | Dipropylene | 2 | 2 | 1 |
| | glycol | | | |
| | 1,2-hexanediol | 2 | 2 | 2 |
| | EDTA-2Na | 0.05 | 0.05 | 0.05 |
| | Allantoin | 0.01 | 0.01 | 0.01 |
| | Ethyl ascorbic | 0.2 | 0.2 | 0.2 |
| | acid | | | |
| | Turmeric root | 0.1 | 0.1 | 0.1 |
| | extract | | | |
| | Purified water | 52.65 | 48.21 | 39.52 |
| Phase D | Potassium | 0.14 | 0.23 | 0.25 |
| (neutralizing | hydroxide | | | |
| agent) | Purified water | 3 | 3 | 3 |
| Phase E | Pine needle | 0.05 | 0.05 | 0.05 |
| (additives) | extract | | | |
| | Pomegranate | 0.1 | 0.1 | 0.1 |
| | Extract | | | |
| | Pine mushroom | 0.1 | 0.1 | 0.1 |
| | extract | | | |
| | Lavender citrus | 0.05 | 0.05 | 0.05 |
| | fragrance | | | |
| | Quercetin | 0.05 | 0.05 | 0.05 |

| | | | |
|---|---|---|---|
| Total | 100 | 100 | 100 |
| Appearance | LOW- | LOW- | LOW- |
| | viscosity | viscosity | viscosity |
| | emulsion | emulsion | emulsion |
| pH | 5.72 | 5.68 | 5.83 |
| Average particle size (nm) | 1.67 | 2.23 | 2.75 |
| Specific gravity | 0.987 | 0.985 | 0.982 |
| Stability (for 3 months | Stable | Stable | Stable |
| at 45° C.) | | | |

*Preparation method: 1) Phase A was weighed, followed by dissolution while heating to 78 to 85° C. 2) Phase B separately weighed and well dispersed and dissolved was mixed with Phase C, followed by stirring and dissolving while heating to 78 to 85° C. 3) Phase A, Phase B and Phase C were mixed and stirred with a homogenizer at 4,000 rpm for 5 minutes to obtain an emulsion. 4) Phase D was added thereto, followed by stirring with a homogenizer at 4,000 rpm for 3 minutes to perform neutralization. 5) The result was cooled to 45° C. and Phase E was added, followed by mixing while stirring. 6) The result was cooled to 30° C., followed by vacuum defoaming to complete preparation.

### Examples 50 to 52: Preparation of Cosmetic Composition (Moisturizing Cream) Containing Vegetable Surfactant of the Present Invention

In this example, a cosmetic composition containing the vegetable surfactant of the present invention (Example 3) was prepared. For example, moisturizing cream was prepared and the detailed composition thereof is shown in Table 16 below.

TABLE 16

| | | Example | Example | Example |
|---|---|---|---|---|
| Component name | | 50 (wt %) | 51 (wt %) | 52 (wt %) |
| Phase A | Example 3 | 3 | 5 | 10 |
| (oily | (vegetable | | | |
| phase) | surfactant) | | | |
| | Cetostearyl | 5 | 5 | 5 |
| | alcohol | | | |
| | Behenyl alcohol | 0.5 | 1 | 1.5 |
| | Microcrystalline | 0.8 | 1 | 1.2 |
| | wax | | | |
| | Beeswax | 1 | 1 | 1 |
| | Cetyl | 5 | 8 | 10 |
| | ethylhexanoate | | | |
| | Ethylhexyl | 3 | 2 | 3 |
| | palmitate | | | |
| | Argan oil | 1 | 1 | 1 |
| | Olive oil | 1 | 0.8 | 0.6 |
| | Tocopheryl | 0.1 | 0.1 | 0.1 |
| | acetate | | | |
| | Ascorbyl | 0.1 | 0.1 | 0.1 |
| | tetraisopalmitate | | | |
| | Camellia oil | 0.8 | 0.5 | 0.6 |
| Phase B | Carbomer-940 | 0.32 | 0.36 | 0.4 |
| (Aqueous | Transparent | 0.05 | 0.5 | 0.05 |
| phase 1) | xanthan gum | | | |
| | Purified water | 25 | 25 | 25 |
| Phase C | glycerin | 5 | 5 | 5 |
| (Aqueous | Dipropylene | 3 | 3 | 3 |
| phase 2) | glycol | | | |
| | 1,3-Butylene | 2 | 2 | 2 |
| | glycol | | | |
| | 1,2-hexanediol | 2 | 2 | 2 |
| | Ethyl | 0.1 | 0.1 | 0.1 |
| | hexylglycerin | | | |
| | EDTA-2Na | 0.05 | 0.05 | 0.05 |
| | Allantoin | 0.1 | 0.1 | 0.1 |
| | Ethyl ascorbic | 0.5 | 0.8 | 1 |
| | acid | | | |
| | Turmeric root | 0.1 | 0.1 | 0.1 |
| | extract | | | |
| | Purified water | 34.9 | 29.91 | 21.52 |
| Phase D | Potassium | 0.1 | 0.1 | 0.1 |
| (Neutralizing | hydroxide | | | |
| agent) | Purified water | 5 | 5 | 5 |
| Phase E | Bamboo extract | 0.1 | 0.1 | 0.1 |
| (additives) | Cucumber extract | 0.1 | 0.1 | 0.1 |
| | Carrot extract | 0.1 | 0.1 | 0.1 |
| | Fragrance | 0.05 | 0.05 | 0.05 |
| | retinol | 0.08 | 0.08 | 0.08 |
| | Acetyl | 0.05 | 0.05 | 0.05 |
| | hexapeptide-8 | | | |

| | | | |
|---|---|---|---|
| Total | 100 | 100 | 100 |
| Appearance | High- | High- | High- |
| | viscosity | viscosity | viscosity |
| | emulsion | emulsion | emulsion |
| Phase | Cream | Cream | Cream |
| pH | 5.86 | 5.95 | 6.02 |
| Average particle size (nm) | 3.12 | 3.68 | 3.96 |
| gravity | 0.983 | 0.982 | 0.981 |
| Stability (for 3 months at | Stable | Stable | Stable |
| 45° C.) | | | |

*Preparation method: 1) Phase A was weighed, followed by dissolution while heating to 78 to 85° C. 2) Phase B separately weighed and well dispersed and dissolved was mixed with Phase C, followed by stirring and dissolving while heating to 78 to 85° C. 3) Phase A, Phase B and Phase C were mixed and stirred with a homogenizer at 4,000 rpm for 5 minutes to obtain an emulsion. 4) Phase D was added thereto, followed by stirring with a homogenizer at 4,000 rpm for 3 minutes to perform neutralization. 5) The result was cooled to 30° C., followed by vacuum defoaming to complete preparation.

### Examples 53 to 55: Preparation of Cosmetic Composition (Ampoule Essence) Containing Vegetable Surfactant of the Present Invention

In this example, a cosmetic composition containing the vegetable surfactant of the present invention (Example 3) was prepared. For example, ampoule essence was prepared and the detailed composition thereof is shown in Table 17 below.

TABLE 17

| | | Example 53 | Example 54 | Example 55 |
|---|---|---|---|---|
| Component name | | (wt %) | (wt %) | (wt %) |
| Phase A | Example 3 | 1 | 3 | 5 |
| (oily | (vegetable | | | |
| phase) | surfactant) | | | |
| | Dimethicone | 5 | 5 | 5 |
| | Cetyl | 1 | 2 | 3 |
| | ethylhexanoate | | | |
| | Ethylhexyl | 1 | 1 | 1 |
| | palmitate | | | |
| | Argan oil | 1 | 1 | 1 |
| | Retinol 50C | 0.08 | 0.08 | 0.08 |
| | Olive oil | 0.8 | 0.8 | 0.8 |
| | Tocopheryl | 0.1 | 0.1 | 0.1 |
| | acetate | | | |
| | Camellia oil | 0.5 | 0.5 | 0.5 |
| Phase B | Carbomer-940 | 0.2 | 0.25 | 0.3 |
| (aqueous | Transparent | 0.05 | 0.05 | 0.05 |
| phase 1) | xanthan gum | | | |
| | Purified water | 20 | 20 | 20 |
| Phase C | 1,3-Butylene | 3 | 3 | 3 |
| (aqueous | glycol | | | |
| phase 2) | Glycerin | 5 | 5 | 5 |
| | 1,2-hexanediol | 2 | 2 | 2 |
| | Dipropylene | 3 | 3 | 3 |
| | glycol | | | |
| | Idebenone | 0.1 | 0.1 | 0.1 |
| | Ascorbic acid | 0.1 | 0.2 | 0.5 |
| | Ethyl ascorbic | 0.5 | 0.3 | 0.2 |
| | acid | | | |
| | Genistein | 0.1 | 0.1 | 0.1 |
| | Purified water | 51.96 | 48.98 | 45.71 |
| Phase D | Potassium | 0.15 | 0.18 | 0.2 |
| | hydroxide | | | |
| | Purified water | 3 | 3 | 3 |
| Phase E | Beta-glucan | 0.1 | 0.1 | 0.1 |
| (additives) | Hibiscus | 0.1 | 0.1 | 0.1 |
| | extract | | | |
| | Rose extract | 0.1 | 0.1 | 0.1 |
| | Sodium | 0.01 | 0.01 | 0.01 |
| | hyaluronic acid | | | |
| | Fragrance | 0.05 | 0.05 | 0.05 |

| | | | |
|---|---|---|---|
| Total | 100 | 100 | 100 |
| Appearance | LOW- | Low- | LOW- |
| | viscosity | viscosity | viscosity |
| | emulsion | emulsion | emulsion |
| Phase | Milky | Milky | Milky |
| | white | white | white |
| pH | 5.85 | 5.89 | 5.82 |
| Average particle size (nm) | 1.58 | 1.96 | 2.13 |
| gravity | 1.001 | 0.999 | 0.998 |
| Stability (for 3 months | Stable | Stable | Stable |
| at 45° C.) | | | |

*Preparation method: 1) Phase A was weighed, followed by dissolution while heating to 78 to 85° C. 2) Phase B separately weighed and well dispersed and dissolved was mixed with Phase C, followed by stirring and dissolving while heating to 78 to 85° C. 3) Phase A, Phase B and Phase C were mixed and stirred with a homogenizer at 4,000 rpm for 5 minutes to obtain an emulsion. 4) Phase D was added thereto, followed by stirring with a homogenizer at 4, 000 rpm for 3 minutes to perform neutralization. 5) The result was cooled to 45° C. and Phase E was added, followed by mixing while stirring. 6) The result was cooled to 30° C., followed by vacuum defoaming to complete preparation.

As is apparent from the foregoing, the present invention provides a vegetable mixed surfactant that has both solubilizing and emulsifying power based on a combination of high-purity polyglyceryl-10 oleate and polyglyceryl-10 stearate without adding ethylene oxide. In this case, the vegetable mixed surfactant is capable of excellent esterification reaction because it contains arginine and secures long-term stability without causing changes in odor or color because it contains tranexamic acid. The vegetable mixed surfactant has excellent skin stability, very low cytotoxicity, excellent short-term moisturizing effect and long-term moisturizing effect, and superior skin improvement effects such as fine wrinkle amelioration effect, skin whitening effect, and skin lifting improvement effect. The vegetable mixed surfactant may be applied to cosmetic compositions of various formulations (skin toner, hair toner, transparent essence, emulsion, cream, and like).

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of preparing a vegetable mixed surfactant comprising:
dissolving a mixture of oleic acid and stearic acid as a lipophilic moiety in polyglyceryl-10 as a hydrophilic moiety (a);
adding arginine to the resulting product in the presence of nitrogen to induce esterification at a pH of 9 to 12.5 (b);
adjusting the pH to 6 to 8 using a pH adjuster (c); and
stirring the result along with a stabilizer (d).

2. The method according to claim 1, wherein in step (b), the esterification is performed twice.

3. The method according to claim 1, wherein the stabilizer comprises at least one selected from tranexamic acid, tocopheryl acetate, ferulic acid, catechin, and baicalin.

4. A micelle formed by self-assembly of the vegetable mixed surfactant obtained by the method according to claim 1.

5. The micelle according to claim 4, wherein the micelle has a solubilizing power to provide a transparent phase and an emulsifying power.

6. The micelle according to claim 4, wherein the micelle comprises a lipophilic substance trapped therein.

7. A cosmetic composition comprising the micelle according to claim 4.

8. A method for improving skin wrinkles or skin elasticity, or moisturizing skin, the method comprising administering the cosmetic composition according to claim 7 to skin of a subject in need thereof.
